(19) United States
(12) Patent Application Publication
(10) Pub. No.: US 2012/0116300 A1
(43) Pub. Date: May 10, 2012

(54) ACTIVATION AND DELIVERY DEVICES FOR THERAPEUTIC COMPOSITIONS

(76) Inventors: Samuel D. Banks, JR., Lexington, MA (US); Katie A. Bush, Boston, MA (US); Thomas D. Egan, Marblehead, MA (US); Braden C. Fleming, East Greenwich, RI (US); Martha M. Murray, Sherborn, MA (US); Matthew Palmer, Cambridge, MA (US); David M. Skinlo, Logan, UT (US); Thomas P. Weisel, Ventura, CA (US); Emma Wright, Buckinghamshire (GB)

(21) Appl. No.: 12/998,854

(22) PCT Filed: Dec. 9, 2009
(86) PCT No.: PCT/US09/67380

§ 371 Date: Jan. 17, 2012

Related U.S. Application Data
(60) Provisional application No. 61/154,181, filed on Feb. 20, 2009.
(60) Provisional application No. 61/154,981, filed on Feb. 24, 2009.
(60) Provisional application No. 61/154,975, filed on Feb. 24, 2009.
(60) Provisional application No. 61/169,062, filed on Apr. 14, 2009.
(60) Provisional application No. 61/228,657, filed on Jul. 27, 2009.

(51) Int. Cl. A61M 5/00

(52) U.S. Cl. 604/82

(57) ABSTRACT

Devices and methods for activating a therapeutic composition, delivering a therapeutic composition to a specific region or tissue in a body, and containing a therapeutic composition within a body cavity are disclosed herein. In one embodiment, a therapeutic capsule is shown having an outer element defining an interior chamber and including a matrix disposed therein and wherein the interior chamber is elongated along a chamber axis.

### RELATED APPLICATIONS

This application claims priority to the following U.S. Provisional patent applications: U.S. Ser. No. 61/121,113, filed Dec. 9, 2008; U.S. Ser. No. 61/154,181, filed Feb. 20, 2009; U.S. Ser. No. 61/154,981, filed Feb. 24, 2009; U.S. Ser. No. 61/154,975, filed Feb. 24, 2009; U.S. Ser. No. 61/169,062, filed Apr. 14, 2009; U.S. Ser. No. 61/224,295, filed Jul. 9, 2009; and U.S. Ser. No. 61/228,657, filed Jul. 27, 2009.

### FIELD

The present invention relates to devices and methods for containing, preparing, delivering and maintaining the effects of a therapeutic composition for healing, repairing, or enhancing tissue in a body. The invention finds particular utility in preparing, delivering and maintaining therapeutic effects of a composition in body locations accessed by minimally invasive surgery (MIS) techniques. While embodiments of the present invention have application in providing therapeutic effects to a wide array of tissues in an animal body, applications are described herein involving healing, repairing and enhancing of connective tissues in the human musculoskeletal system for illustrative purposes. In particular, applications to soft tissues that are exposed to synovial fluid, such as the anterior cruciate ligament (ACL) of the knee and the rotator cuff tendons (RC) of the shoulder are described.

### BACKGROUND

Most tissues in the body heal spontaneously when damaged. Other tissues, particularly intra-articular tissue, such as the anterior cruciate ligament (ACL) for example, fails to heal adequately after rupture, even after partial injuries or primary repair with sutures or other fixation devices. This phenomenon is quite different from the response of extra-articular injuries such as those to the medial collateral ligament (MCL) which often heals even with no surgical intervention. The lack of healing seen in the ACL and other intra-articular tissues has been attributed to the relative lack of vascularity of ACL's, the hostile environment of synovial fluid, alterations in the cellular metabolism after injury, intrinsic cell deficiencies, and the complex biomechanics of these tissues. However, recent publications have reported that human ACL remnants typically contain viable cells and vasculature, yet there is a gap at the rupture site which remains open. This gap is unique to the intra-articular environment of the ACL as it is not observed in repaired extra-articular ligaments.

In a typical extra-articular MCL wound site, the gap resulting from rupture is filled with a provisional scaffold of fibrin and platelets that is subsequently invaded by surrounding intrinsic and extrinsic cells which initiate and conduct tissue healing. In the extra-articular environment the breakdown of this fibrin-platelet provisional scaffold occurs slowly over days to weeks in balance with new collagen formation at the injury site. In contrast, in the intra-articular environment of the ACL, such a stabilized fibrin-platelet clot is not observed to form even after induced bleeding into the joint. Thus, patients with an intra-articular injury form a hemarthrosis in the joint, without forming a stabilized fibrin-platelet scaffold at the wound site. The presence of urokinase plasminogen activator found in the synovial fluid after injury may play a key role in this failure of a scaffold formation as the presence of this enzyme results in elevated levels of circulating plasmin in the joint and accelerated fibrin dissolution. Other intra-articular soft tissues in the body experience a similar resistance to healing to various degrees, including the meniscus and posterior collateral ligament (PCL) of the knee, the rotator cuff tendon and glenoid labrum in the shoulder, the labrum of the hip, and the triangular fibrocartilage complex (TFCC) of the wrist.

In addition to repair of native tissue, reconstruction of tissue in an animal by means of a graft is known to the art. Numerous examples of replacement or augmentation of weak or damaged ligament, tendon, cartilage, bone, muscle, vascular, nerve and organ tissues (hereafter “tissue”) with autograft, allograft, xenograft (hereafter “tissue graft”) or synthetic graft materials in the body have been demonstrated or hypothesized. All are potential beneficiaries of the present invention. Particular applicability can be seen in the use of tissue grafts to buttress, replace, or augment (hereafter “reconstruct”) ligament, tendon, cartilage, and fibrocartilagenous tissue (hereafter “connective tissue”) in the knee, shoulder, elbow, wrist, hip, ankle (“joints”) and spine. In practice, a reconstruction is rarely as strong as natural, healthy, uncompromised tissue. Animal studies show reconstruction grafts of the ACL, by example, typically restore only about 40% of pre-injury strength, putting the patient at risk of re-injury and early onset of degenerative joint disease. The graft initially has no blood supply and the original graft tissue degrades over time while at the same time it is being repopulated with new living cells which can remodel the graft and lay down new tissue. The need exists for materials and methods to enhance and accelerate the cellular remodeling process, thereby improving graft characteristics such as strength, laxity and patient recovery time.

To address this problem of non-healing soft tissues, certain therapeutic compositions have been developed to improve healing, integration, and remodeling of the newly implanted tissue graft. Recent studies have shown that new cellular growth in connective tissue can be brought about through the introduction of a composition of collagen, platelets, and at least one of an extracellular protein and a neutralizing agent, and subsequent related compounds. Other compounds include any combination of collagen (all types), proteoglycans, glycosaminoglycans, carbohydrates, synthetic material, (hereafter “matrix”) and one or more biologically active agents such as blood, plasma, platelet rich plasma, platelet poor plasma, bone marrow aspirate, stem cells, growth factors, proteins, peptides, etc. (hereafter “biologically active agents”), and in some cases inert ingredients such as gelling agents, foaming agents, gelatin, gelatin foam, cellulose, preservatives, textures, binders, etc. (hereafter “inert ingredients”). Collectively, these combinations of ingredients in any combination shall be referred to as “therapeutic compositions”.

By applying these therapeutic compositions to damaged soft intra-articular tissue, one can create a provisional scaffold, a stabilized clot can be simulated that is subsequently invaded by surrounding intrinsic and extrinsic cells which initiate and conduct tissue regeneration. Breakdown of this fibrin-platelet provisional scaffold occurs slowly over days to weeks in balance with new collagen formation strengthening the repair.

The physical configuration of the repair may take any of several forms. The repair may involve abutting or apposing two ends of torn tissue such as a ligament. In situations where the soft tissue to be repaired is load bearing, and immobilization of the patient is undesirable or impractical, a temporary fixation means, such as strands of suture, may be used to hold the soft tissue in apposition until healing can take place. Whether the soft tissue tear is within the middle portion of the soft tissue, or at the ends where it interfaces with other tissues (bone, muscle, etc.), the therapeutic composition can be placed around or throughout the defect to enhance healing in those regions. The repair may also involve bridging a gap of missing or severely compromised soft tissue where it is not possible to bring together the desired tissues. In these cases the therapeutic composition can be placed around the soft tissues and inside the gap between these tissues in order to allow cells to migrate into the gap between the two desired fixation points. Still other soft tissue injuries call for enhancement of stretched, partially torn or only weakened tissue. Additionally, therapeutic compositions may be used to improve biomechanical qualities of a tissue graft used to reconstruct native anatomy. It is therefore desirable to have a means to place a therapeutic composition against or around a tissue structure or to fill a gap in tissue or to fill a gap transited by suture or other repair structures.

Amorphous therapeutic compositions that assume the shape of a body cavity into which they are delivered, are in contact with tissues enclosing the body cavity. Often the targeted tissue or injury is present in only a small region of the body cavity. When the therapeutic effects of the composition are delivered through contact, and only a small percentage of the tissues defining the body cavity are targeted for therapeutic effects, a large percentage of the therapeutic effects can be delivered to healthy tissue in no need of healing. For example, a torn ACL of the knee could be treated by delivery of a therapeutic composition to the space surrounding the ACL (the intracondylar notch). An amorphous therapeutic composition could be injected into the intracondylar notch to surround and envelop the ACL, imparting therapeutic benefits through direct contact with the injured tissue. However, the composition would also be in direct contact with the posterior cruciate ligament (PCL), the cortical surface of the femur, the articular cartilage of the femoral condyles and tibial plateau, and the meniscus. It would be more efficient to deliver therapeutic composition only to the specific tissue to be treated. Thus, there is a need in some cases to contain the therapeutic composition in a region around a target tissue.

### SUMMARY

Devices and methods for activating a therapeutic composition, delivering a therapeutic composition to a specific region or tissue in a body, and containing a therapeutic composition within a body cavity are disclosed herein.

An embodiment of the device includes a hollow chamber containing a matrix, which may be an embodiment of a neutralized soluble extracellular matrix. In a preferred embodiment, the matrix is a dry porous sponge. In other embodiments, the matrix is a powder, solid, liquid, or gel. The device includes a means for adding a biologically active agent to the matrix to form a therapeutic composition. In a preferred embodiment, the biologically active agent is a liquid. In other embodiments, the biologically active agent is a powder, solid, liquid, or gel of a type capable of being mixed with the particular form of the matrix. In an embodiment of the invention, the biologically active agent is whole blood, platelet-rich plasma (PRP), or saline.

In an embodiment, the chamber containing the matrix consists of a fully enclosed shell of biocompatible, bioabsorbable material, such as a gelatin capsule, sugar, cellulose, absorbable polymer, or similar material. The chamber is penetrated with a hypodermic type needle and injected with a quantity of a biologically active agent to activate the matrix and form a therapeutic composition. The chamber or capsule is placed in a part of the body, adjacent to the tissue to be treated and when the capsule dissolves or is absorbed, the therapeutic composition is released, treating the targeted tissue.

Alternatively the chamber or capsule may be placed in the body prior to activation and be activated by needle injection in situ. Other embodiments include a tube attached to the chamber communicating with the matrix, allowing delivery of a biologically active agent remotely. Still other embodiments are empty chambers with means for filling with therapeutic composition in situ. Some of these embodiments are collapsible to allow introduction to the body through a small opening, as in minimally invasive surgery (MIS). Other embodiments are not fully enclosed but have openings into which tissue to be treated may be placed.

In a preferred embodiment, the chamber and matrix take the form of a hollow sleeve with a therapeutic composition matrix attached to its inner surface that may be slid over a linear tissue structure, such as an ACL graft, such that the therapeutic composition is held in direct contact with the graft. In some embodiments, this sleeve is slid over the graft prior to placement in the body, or after one end of the graft is fixed in place. In other embodiments the chamber and matrix is split such that it can be wrapped around the graft or linear tissue structure. Other embodiments of the implantable chamber include provision for sutures to be passed through the chamber to facilitate delivery to a targeted tissue or to fix the therapeutic composition in place in a body.

In another class of embodiments, the chamber containing the therapeutic composition is removed from the body after delivery of the therapeutic composition to the targeted tissue. The material of the chamber therefore need not be dissolvable or absorbable. This embodiment is particularly useful for therapeutic compositions that transition from a relatively amorphous state to a semi-solid state, and in environments like arthroscopic surgery where the saline fluid filled joint may fragment or dissolve the therapeutic composition when in its amorphous state. In this case the chamber provides a protective environment for delivery of the therapeutic composition and is removed when the material transitions to a gel-like state that is stable in the joint environment.

In other embodiments of the present invention, the chamber itself never enters the body, and only the therapeutic composition is delivered to the target tissue. In this class of embodiments, the chamber containing the matrix receives a biologically active agent through a passageway in communication with the matrix, and the activated therapeutic composition is ejected from the device into the body through a hollow delivery channel.

In an embodiment, the chamber is open at one end and a piston-like pusher ejects the activated therapeutic composition from the distal end of the device directly into a body opening. This and other embodiments may or may not have provisions for threading guiding/fixating sutures through the therapeutic composition mass.

In an embodiment, the chamber is substantially cylindrical with a proximal and a distal end, and the matrix is an elongated shape with an outside diameter, an inside diameter, and a length. In this embodiment, the passageway that delivers the biologically active agent is a tube that extends axially through the length of the chamber, through the inside diameter of the matrix, terminating near the distal end of the chamber. In this embodiment a piston moves the matrix axially toward the distal end while simultaneously another connected mechanism pumps a biologically active agent through the passageway toward the distal end of the chamber at a rate proportional to the movement of the matrix. In this way, the dry, sponge-like matrix is wetted from the inside with a progressively as it travels down the cylindrical chamber, and exits the cylindrical chamber as an activated therapeutic composition. The proportional relationship of the matrix advancing mechanism and the biologically active agent dispensing mechanism assure the proper ratios of each ingredient are maintained.

In an embodiment, the matrix is advanced as a single elongated piece, which preferably is a tube. In another embodiment, the matrix is comprised of a plurality of shorter tube segments. In another embodiment, the passageway introducing the biologically active agent does not extend axially through the center of the cylindrical chamber, but instead exists outside the lumen of the cylindrical chamber, communicating with the chamber near the distal end from the side wall of the chamber. In this embodiment, the matrix need not be tubular, but is instead cylindrical or otherwise conforming to the shape of the chamber. In another embodiment, the matrix segments of this type are loaded individually into the chamber from a magazine and advanced individually, while still maintaining the proportional relationship of matrix-to-biologically-active agent.

Mixing of the matrix and the biologically active agent to form the activated therapeutic composition may be accomplished in a variety of ways depending, on the nature of the matrix and agent. Where both are a liquid or gel, they may be mixed in the chamber using a stirring device, such as an impeller or screw auger. In an embodiment, the liquid or gel matrix and agent are mixed in a chamber using a helical screw auger that is axially compressible. A piston provides a means of propelling the activated therapeutic composition from the chamber, through a delivery channel and into a body. The helical auger compresses within the chamber allowing the piston to displace the volume therein.

In some embodiments, where the matrix is a dry, porous sponge and the biologically active agent is an aqueous liquid, the mixing process required for activation is facilitated by an inherent hydrophilic absorbency of the sponge; the matrix becomes fully saturated by coming into contact with the appropriate volume of a biologically active agent. In other embodiments, with less hydrophilic matrices, it is necessary to provide a means of inducing the two or more ingredients to thoroughly mix.

One means of inducing wetting is removal of air from the porous matrix, wetting in a vacuum, and then re-pressurizing to force the liquid into the pores of the sponge. In an embodiment, the chamber containing the dry matrix is evacuated of air and sealed, such that the matrix exists in a full or partial vacuum. A passageway for the introduction of a biologically active agent is provided but sealed until needed for activation (immediately prior to delivery to a body.) During activation, the biologically active agent is applied to the matrix in a vacuum and the chamber is re-pressurized by allowing atmospheric pressure air into the chamber, forcing the liquid agent onto the pores of the sponge matrix. In another embodiment, the chamber is re-pressurized by changing the volume of the chamber to equalize pressure in the chamber with the surrounding atmosphere. In an embodiment, this is accomplished by providing a matrix in a cylindrical chamber with a piston. The piston is locked in a retracted position prior to evacuation of the chamber. A volume of a biologically active agent, calculated to fully saturate the matrix, is introduced into the chamber without allowing the admittance of air. At this point, the chamber contains partially wetted matrix sponge, liquid agent, and empty space in the form of sponge pores in a vacuum. Without allowing air into the chamber, the lock holding the piston in a retracted position is released and the piston is allowed to slide into the chamber, propelled by the pressure differential between the chamber (vacuum) and the surrounding atmosphere. As the piston slides into the chamber space, the evacuated pores are collapsed or filled with liquid agent until the piston stops moving and the pressure inside and outside the chamber is equalized. The resulting activated therapeutic composition has little or no entrapped air.

An airless activated therapeutic composition has several advantages over activated therapeutic compositions with entrapped air: (i) it is roughly neutrally buoyant in the saline irrigated environment of arthroscopic surgery and will not float away; (ii) when extruded through a hollow delivery channel, it will not sputter and fragment like composition with air will; (iii) it is incompressible and will not compress and admit saline back flow when the delivery channel and chamber are exposed to pressurized arthroscopic saline; and (iv) it contains a higher concentration per unit volume of therapeutic agents than composition with entrapped air.

In another embodiment, rather than evacuating and sealing the chamber at the time of manufacture, a means for evacuating the chamber at the time of use is provided. In this embodiment, a vacuum source (a vacuum pump, an evacuated container, etc.) is connected to the matrix-containing chamber by a passageway equipped with a 3-way valve. In a first position, the valve connects the chamber to the atmosphere. In a second position, the valve connects the chamber to the vacuum source. In a third position, the valve connects the chamber to a reservoir containing a quantity of biologically active composition. By stepping through the valve positions, first, second, third, then back to first, the matrix has been vacuum activated. Alternatively, when a chamber with a locking piston is used, valve steps first, second, and third are followed by release of the piston lock, rather that switching the valve back to the first, vented, position.

In some embodiments of the therapeutic composition, the matrix and biologically active agent are mixed at or near the time of their application to the repair site. This mixing process is referred to as “activation” of the therapeutic composition. In some instances, the matrix is a dry, absorbent substance like a sponge, and the biologically active agent is a liquid-based composition applied to the matrix immediately before, after, or during implantation to form the therapeutic composition. In some instances, the matrix is wetted with biologically active agent and assumes a flowable, amorphous state with a paste-like consistency where it is able to be delivered by injection or extrusion into a body using a device like a syringe.

Once in the body, the amorphous material assumes the shape of the body cavity it is injected or extruded into. Some embodiments of the therapeutic composition transform in the body from an amorphous state to a non-flowable, semi-solid state, like a gel, foam, or solid. Other embodiments of the therapeutic composition retain their amorphous state after delivery. Some embodiments of the therapeutic composition that transition from an amorphous, paste-like consistency to a semi-solid, gel-like consistency do so rapidly after activation by mixing matrix and active agent. In these embodiments, it is desirable to activate the composition immediately prior to delivery or even during the delivery process to prevent such composition transition prior to reaching the region of tissue to be healed.

Other embodiments include a dry, porous matrix, such as a collagen sponge, which is activated by mixing with a liquid biologically active agent, such as platelets, blood, or platelet-rich plasma (PRP), and where the resulting amorphous therapeutic composition is to be delivered to a target site by being pushed, injected, or extruded through a tube. In such instances, it is often desirable to remove air from the mixture to assure faster, more complete wetting, increase the density and concentration of the resulting composition, prevent back-flow of irrigation fluid due to compressibility of air-filled material, and prevent sputtering and fragmentation of the extruded material due to air entrapment.

An embodiment of the present invention includes a therapeutic capsule having at outer element defining an interior chamber and including a matrix disposed therein. In an embodiment, the interior chamber is elongated along a chamber axis, and the matrix includes a cylindrical central void region having a diameter D and extending from at least a first end thereof and along the chamber axis.

Another embodiment of the present invention includes a therapeutic kit, comprising a therapeutic capsule, having at outer element defining an interior chamber and including a matrix, such as a collagen matrix, disposed therein, together with an introducing device adapted to introduce a fluid biologically active agent to the matrix. In one embodiment, the introducing device of the kit includes a cylindrical elongated delivery element disposed about a central void region extending along a delivery axis from a proximal end to a distal end thereof. The elongated delivery element further includes a cylindrical outer surface extending from the distal end along the delivery axis, and has a diameter less than or equal to D. In one embodiment, the central void region of the elongated delivery element is adapted at the proximal end to receive the fluid biologically active agent. The delivery element may include one or more radially extending holes near the distal end and extending between the outer surface and the central void region.

In another embodiment of the therapeutic kit of the present invention, the introducing device is a syringe, having an elongated reservoir element disposed about a reservoir region and extending from a proximal end to a distal end along the delivery axis. In an embodiment, the introducing device further includes a piston, disposed in the reservoir element near the proximal end, and a cap at the distal end thereof, wherein the reservoir region is disposed between the piston and the cap, and wherein the cylindrical elongated delivery element extends from the cap, whereby the central void region is in fluid communication with the reservoir region.

In another embodiment of the therapeutic capsule of the present invention, the outer element defines the chamber to be closed and fully enclosing the matrix. In an alternative embodiment, the outer element defines the chamber to be open-ended, having a first open end along the chamber axis at one end thereof, and a second open end along the chamber axis opposite thereto. The outer element may include a longitudinal slit therein, wherein the matrix is longitudinally segmented, and wherein each segment extends radially inward from the outer element and is tapered in a radial direction. In another embodiment, the outer element of the therapeutic capsule includes one or more through-holes extending radially therethrough.

In an embodiment of the invention, the outer element is longitudinally segmented into a plurality of segments extending in a direction substantially parallel to the chamber axis.

In an embodiment of the invention, the power element is longitudinally segmented into two segments, each of the segments having C-shaped cross-sections along the chamber axis.

In an embodiment of the therapeutic capsule of the present invention, the outer element is bioabsorbable. In alternative embodiments, the outer element is composed of one from the group consisting of gelatin, sugar, cellulose, and polymer. In yet other embodiments, the outer element includes an elongated extension extending therefrom, wherein the elongated extension defines an interior region in fluid contact with the chamber.

In an embodiment of the therapeutic capsule, the elongated extension is flexible. In another embodiment, the matrix is compressible and the outer element is flexible. In an alternative embodiment, the matrix is dry and porous. In an embodiment, the dry porous matrix is a sponge.

In an embodiment of the inventive therapeutic kit, the kit further comprises an introducing device adapted to introduce a fluid biologically active agent to the matrix. In an embodiment, the introducing device is a syringe. In an embodiment of the kit, the biologically active agent comprises platelet-rich plasma (PRP) or blood. In an embodiment of the inventive kit, the matrix is responsive to introduction of the PRP or blood to become amorphous. In alternative embodiments, the biologically active agent may be PPP, whole blood, or platelets.

One embodiment of an activation device for a therapeutic compound of the present invention, an outer element defines an interior chamber and includes a dry porous matrix, such as a collagen matrix, disposed therein, wherein the outer element defines the chamber to be closed and fully enclosing the matrix, wherein the chamber is characterized by a static pressure above or below ambient, and wherein the outer element is adapted to receive a delivery element for infusing said matrix with a biologically active agent.

In an alternative embodiment of the inventive therapeutic kit for a therapeutic compound, the kit further includes an activation device, and an introducing device adapted to introduce a fluid biologically active agent through the outer element and into the matrix.

In an embodiment of an activation device for a therapeutic compound, the device further comprises an outer element defining a cylindrical interior chamber extending along a central axis, the chamber having a piston disposed at one end thereof, and being sealed at the other end thereof. In that embodiment, the chamber includes a dry porous matrix, such as a collagen matrix, disposed therein, further including a selectively operative latch assembly, operative in a first state to fixedly hold, or lock the piston in the chamber, and operable in a second state to release the plunger allowing motion of the plunger in the chamber along the central axis in response to a pressure differential across the plunger, and wherein the chamber is characterized by a static pressure below ambient, and wherein the piston is adapted to receive a delivery element for infusing the chamber with a biologically active agent.

In an embodiment of an inventive introducer for allowing passage of a compressible cylindrical therapeutic capsule disposed on an axial extending suture, the introducer comprises a tube extending along an introducer axis from a proximal end to a distal end, wherein the tube is characterized by a monotonically increasing diameter from the proximal end to the distal end, and wherein the tube included a longitudinal slit extending from the proximal end to the distal end.

In an embodiment of an inventive delivery kit for delivering a therapeutic compound, the compound including a cylindrical, suture-bearing, dry porous matrix, such as a collagen matrix, the kit includes: (i) an outer elongated element having a length L1 and extending from a proximal end to a distal end along a delivery axis and defining an inner cylindrical region; (ii) an inner elongated element having a length L2, where L2 is greater than L1, and disposed slidably within the inner cylindrical region of the outer elongated element, and extending from a proximal end to a distal end along the delivery axis, and defining an inner cylindrical region having diameter D1; (iii) a pusher assembly extending along a pusher axis from a matrix pusher element at a proximal end to a control pusher element at a distal end, wherein at least the matrix pusher element at the distal end is adapted to slide within the inner cylindrical region of the inner elongated element with the pusher axis being substantially coaxial with the delivery axis. In an embodiment of the inventive kit, the matrix pusher element extends transverse to the pusher axis and the pusher element has a circumferential edge has a diameter slightly less than D1 and includes a plurality of void regions extending radially inward from the circumferential edge. In an embodiment of the inventive kit, the control pusher element is rigidly coupled to the matrix pusher element by an intermediate element, and a plurality of elongated suture capture devices, each suture capture device extending along a suture device axis from a handle portion at a proximal end to a suture capture portion at a distal end, and having a length greater than L2.

In an embodiment of a therapeutic composition containment device of the present invention, the device comprises an elongated primary shaft, extending from a proximal end to a distal end along a shaft axis, defining a primary interior region extending along the shaft axis from the proximal end to the distal end. The embodiment further comprises a containment structure extending from the distal end of the elongated shaft, and having two states: (i) a first state, wherein the containment structure is C-shaped and disposed about a central axis transverse to said shaft axis, and extends between opposite ends thereof and defining a gap between said opposite ends; and (ii) a second state, wherein the containment structure is ring-shaped and disposed about the central axis, the ring-shaped containment structure having an innermost surface defining an open faced circular channel extending circumferentially about the central axis, the channel being in fluid communication with the interior region of the shaft.

In an embodiment of a containment device of the present invention, a central axis intersects with and is perpendicular to the shaft axis. In an alternative embodiment, the inventive device includes a state control assembly selectively operable from the proximal end to control the containment structure to be in the first state with the gap being substantially closed, and in the second state with the gap being opened and having a predetermined non-zero value.

In an embodiment of a containment device of the present invention, the device further comprises an outer elongated shaft, disposed along an outer axis and defining an interior region extending along the outer axis, with the outer elongated shaft being adapted to overfit the primary elongated shaft with the outer axis substantially parallel with the shaft axis, whereby the primary elongated shaft can slide within the outer elongated shaft, and wherein the containment structure is compressible to permit positioning within the interior region of the outer elongated shaft.

In an embodiment of an apparatus for loading a therapeutic composition, where the therapeutic composition includes a matrix and a biologically active agent (BAA), the apparatus comprises: (i) a delivery device, defining an interior cylindrical mixing chamber extending along a central axis between a proximal end and a distal end, and including at the distal end, a delivery tube extending therefrom along the central axis from the distal end to a delivery tip, the delivery tube defining an interior region extending along said central axis and having length L and a minimum dimension D transverse to said central axis and being in fluid communication with said Interior mixing chamber; (ii) an activation assembly including a housing having a vacuum port, a biologically active agent (BAA) port, and a loading port and a valve, wherein the vacuum port, the BAA port, and the loading port are coupled to the valve, wherein the valve is operative in a first state, to couple the vacuum port to the loading port, and in a second state, to couple the BAA port to the loading port; and (iii) an elongated mixing tube, coupled to and extending from the loading port, wherein the mixing tube defines an interior BAA flow region extending along a BAA loading axis between the loading port and a distal end of the mixing tube.

In an alternative embodiment of the inventive apparatus, the length of the BAA flow region is greater than L, and the mixing tube includes a plurality of radially-extending holes disposed near the distal end of the mixing tube, with the holes coupling the BAA flow region to points outside the mixing tube, wherein the maximum diameter of the mixing tube is less than D. In an alternative embodiment, a vacuum supply is coupled to the vacuum port. In an alternative embodiment, a BAA reservoir is coupled to the BAA port. In yet another embodiment of the inventive apparatus, a piston is disposed in the cylindrical mixing chamber together with an associated driver, the piston being adapted for motion along the central axis in response to user-controlled action on the driver. In yet another embodiment, the loading port includes a seal for receiving the delivery tip, whereby the interior region of the delivery tube is in fluid communication with the loading port, with the mixing tube disposed within the delivery tube, and with the central axis being substantially coaxial with the BAA loading axis.

In an embodiment of an apparatus of the present invention for loading a therapeutic composition, the therapeutic composition includes a matrix and a biologically active agent (BAA), to a delivery device, whereby the matrix and the BAA are intermixed.

In an embodiment of an activation and dispensing kit for a therapeutic compound, the kit comprises: (i) a biologically active agent (BAA) container defining an interior BAA region extending from a closed proximal end to an elongated, cylindrical open distal end having a diameter R disposed about a BAA axis, and includes a BAA coupler at the open distal end; and (ii) an activator/dispenser. In an embodiment, the activator/dispense includes: a housing; an elongated dispensing tube defining an elongated cylindrical dispensing region extending from the housing to an open distal end along a dispensing axis A; an elongated hollow needle extending from the housing, and defining an interior needle region extending from a proximal end within the housing to a closed distal end within the dispensing tube, wherein the dispensing tube and the hollow needle are coaxial along axis A and define an annular matrix reservoir region in the dispensing region between the dispensing tube and the needle, and wherein the needle includes at or near its distal end, one or more holes coupling the interior needle region with the matrix reservoir region; an elongated matrix pusher having an annular proximal end having an outer diameter greater than R and an extended elongated distal end extending therefrom coaxial along axis A, wherein the distal end is disposed in the matrix reservoir region; a central bore disposed in the matrix reservoir region and extending from an annular proximal end having an outer diameter greater than R; a BAA coupling assembly affixed to the proximal end of the needle, including an annular resilient seal having diameter R and disposed about the proximal end the interior needle region, the BAA coupling assembly being adapted to receive the BAA container, whereby the BAA axis is coaxial with the dispensing axis A and the interior BAA region is fluidly coupled to the interior needle region; and, an actuator assembly, including means for advancing a BAA container coupled to the BAA coupling assembly about the seal, whereby the open end of the BAA container is translated in the direction of the dispensing axis A.

In an alternative embodiment of the inventive kit, the advancing means of the actuator assembly is selectively operative in a first state to advance a BAA container toward the distal end of the dispensing tube and in a second state to advance a BAA container away from the distal end of the dispensing tube. In yet another embodiment of the inventive kit, the advancing means is a selectively bidirectional ratcheting assembly. In yet another alternative embodiment of inventive kit, the one or more holes coupling the interior needle region with the matrix reservoir region, are a plurality of radially extending holes extending from points near the distal end.

### BRIEF DESCRIPTION OF THE DRAWINGS

FIG. 1a is an isometric view of an embodiment of the invention.

FIG. 1b is a partial cutaway isometric view an embodiment of the invention.

FIG. 2 is an isometric view of an embodiment of the invention.

FIG. 3 is an isometric view of an embodiment of the invention.

FIG. 4A is an isometric view of an embodiment of the invention.

FIG. 4B is a cutaway isometric view of an embodiment of the invention.

FIGS. 5a, 5b, and 5c are isometric views of an embodiment of the invention.

FIG. 6a is an isometric view of an embodiment of the invention.

FIG. 6b is a detail partial cutaway isometric view of the embodiment of the invention shown in FIG. 6a.

FIG. 7 is a partial cutaway isometric view of an embodiment of the invention.

FIG. 8 is a partial cutaway isometric view of an embodiment of the invention.

FIG. 9a is an exploded isometric view of an embodiment of the invention.

FIG. 9b is a half-section isometric view of an embodiment of the invention.

FIG. 10a is an exploded isometric view of an embodiment of the invention.

FIG. 10b is an isometric view of the embodiment of the invention shown in FIG. 10a.

FIG. 11a includes both a plan view and a longitudinal half-section view of an embodiment of the invention.

FIG. 11b includes both a plan view and a longitudinal half-section view of an embodiment of the invention.

FIG. 11c includes both a plan view and a longitudinal half-section view of an embodiment of the invention.

FIG. 12 is an isometric view of a prior art bone-tendon-bone autograft.

FIG. 13a is an exploded isometric view of an embodiment of the invention.

FIG. 13b is an isometric view of the embodiment of the invention of FIG. 13a.

FIG. 14 is an isometric view of an embodiment of the invention.

FIG. 15a is an isometric view of an embodiment of the invention.

FIG. 15b is a detail cross-section view of the embodiment of the invention shown in FIG. 15a.

FIG. 16 is an isometric view of an embodiment of the invention.

FIG. 17a is an isometric view of an embodiment of the invention.

FIG. 17b is a detail isometric view of the embodiment of the invention shown in FIG. 17a.

FIG. 18 is an isometric view of a prior art arthroscope.

FIG. 19 is an isometric view of an embodiment of the invention.

FIG. 20a is a detail isometric view of a containment chamber of the invention shown in FIG. 19, in an open position.

FIG. 20b is a detail isometric view of the containment chamber shown in FIG. 19, in a closed position.

FIG. 21a is a detail isometric view of a containment chamber of the invention, in a closed position.

FIG. 21b is a detail isometric view of the containment chamber shown in FIG. 21a, in an open position.

FIG. 22 is an isometric view of an embodiment of the invention.

FIG. 23a is a side plan view of an embodiment of the invention, with the cover removed, and with the trigger lever in a first position.

FIG. 23b is a plan view of the embodiment of the invention shown in FIG. 23a, with the trigger lever in a second position.

FIG. 24 is a side plan view of an embodiment of the invention, with covering removed.

FIG. 25a is an isometric view of an embodiment of the invention with the side covering removed.

FIG. 25b is a detail, partial cutaway section, isometric view of a shaft chamber of the invention.

FIG. 25c is a detail, partial section, isometric view of a shaft chamber of the invention.

FIG. 26a is a detail schematic cross-section of an integral PRP extraction system of the invention.

FIG. 26b is a detail schematic cross-section of one aspect of the integral PRP extraction system of FIG. 26a.

FIG. 26c is a detail schematic cross-section of one aspect of the integral PRP extraction system of FIG. 26a.

FIG. 27a is a side plan view of a therapeutic compound dispensing element of the invention.

FIG. 27b is a hidden line plan view of the therapeutic compound dispensing element shown in FIG. 27a.

FIG. 28a is a side plan view of an embodiment of the invention.

FIG. 28b is a hidden line side plan view of the embodiment of the invention shown in FIG. 28a.

FIG. 29 is an isometric hidden line perspective view of an embodiment of the invention.

FIG. 30a is a schematic of a valve the invention, in a storage position.

FIG. 30b is a schematic of a valve of the invention, in a first position.

FIG. 30c is a schematic of valve of the invention, in a second position.

FIG. 31 is an isometric view of a component of an embodiment of the invention.

FIG. 32 is a longitudinal cross-section view of a chamber of the invention.

FIG. 33 is a longitudinal cross-section view of a chamber of the invention.

FIG. 34a is a longitudinal cross-section view of a chamber of the invention.

FIG. 34b is a longitudinal cross-section view of a chamber of the invention, showing the end cap removed.

FIG. 35 is an isometric view of an embodiment of the invention.

FIG. 36 is an isometric view of an embodiment of the invention.

FIG. 37 is an isometric view of an activated form of the invention shown in FIG. 36.

### DETAILED DESCRIPTION

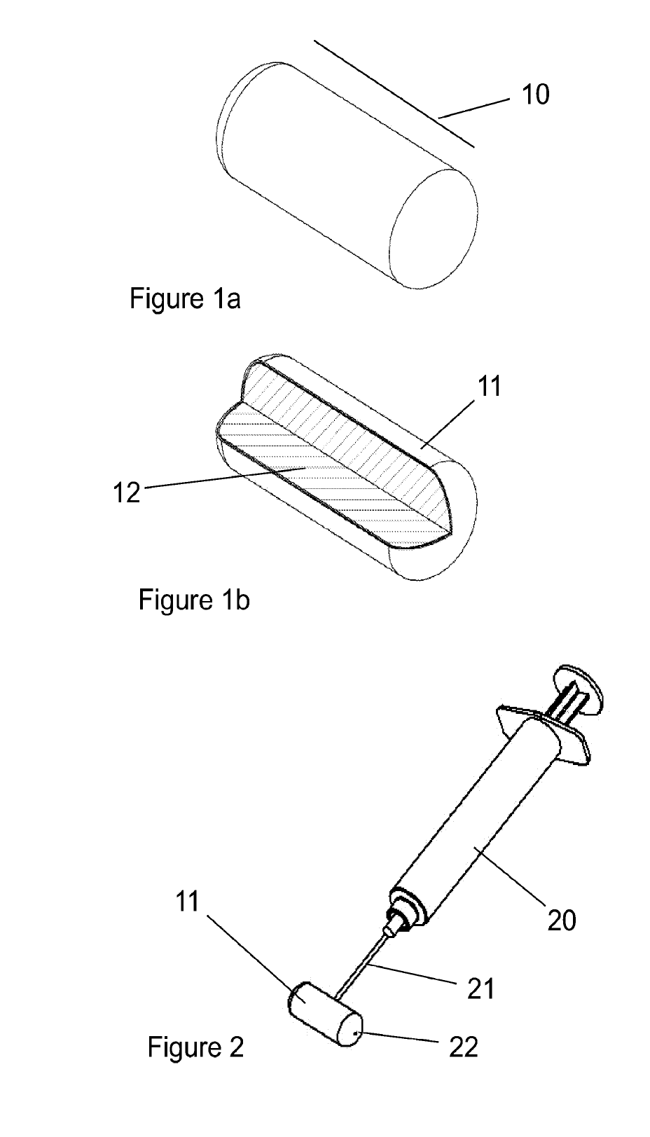

FIG. 1a shows an embodiment of the present invention device 10. FIG. 1b shows a one quarter cutaway view of the embodiment of FIG. 1a showing a chamber 11 containing a matrix 12. In an embodiment the chamber 11 is gelatin. In an embodiment the matrix 12 is a collagen sponge, and preferably may be a soluble type I collagen.

FIG. 2 shows a biologically active agent (BAA) in a syringe 20 being delivered to the matrix in the chamber 11 through a passageway 21 (in this illustrated embodiment, a hypodermic type needle) to form an activated therapeutic composition. In this illustrated embodiment, a small vent hole 22 vents any air or gas in the matrix displaced by the introduction of the biologically active agent. In an embodiment, the biologically active agent is platelet rich plasma (PRP). In alternative embodiments, the agent may be whole blood, saline, or PPP. In this embodiment, the device 10 is then placed in a body adjacent tissue targeted for treatment. When the gelatin chamber dissolves, the activated therapeutic composition comes in contact with the target tissue and provides a therapeutic benefit.

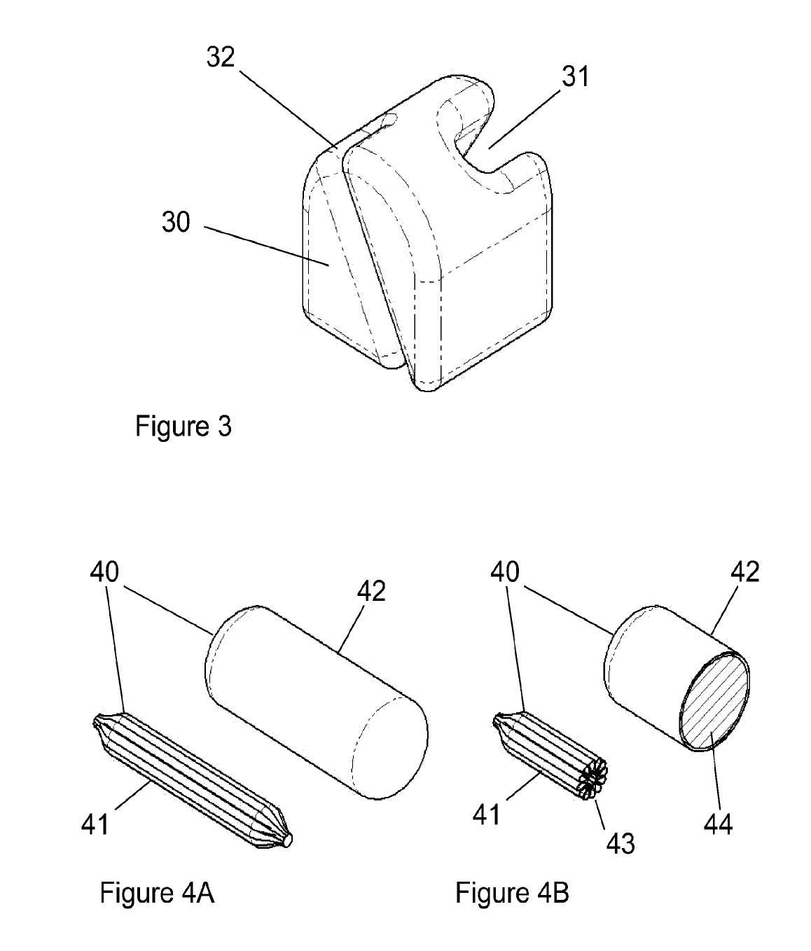

FIG. 3 shows another embodiment of the inventive device 30 comprising a chamber containing a matrix that has been formed in a shape to provide tissue repair to a specific tissue in a specific anatomic region of a body. The illustrated device 30 is designed for use in repairing an ACL located in the femoral condylar notch of the right human knee. In this embodiment, the device 30 is pliable and so the shape need only be approximate. The overall tombstone shape is designed to conform to the condylar notch. A wide diagonal slot 31 conforms to the intact posterior collateral ligament (PCL) and a narrower slot 32 envelops a suture or other structural filament installed to stabilize the joint during the healing process. In one embodiment, the entire surface of the device is covered by the chamber. In another embodiment, the portion of the chamber corresponding to the insertion sites, or attachment points of the repaired ACL, have been removed to directly expose the therapeutic composition to the surfaces to which the repaired ACL will establish in-growth.

FIG. 4A shows two states of another embodiment of the present invention device 40 designed to be delivered to the tissue repair site through a small incision or anatomic passageway. In its collapsed state 41 the device comprises a chamber containing a compressed matrix, pleated and folded into a small diameter, substantially cylindrical shape. Once placed in a body at the tissue repair site, the matrix is activated by the introduction of biologically active agent, thus expanding it into a larger volume state 42. In another embodiment, the pleated chamber is empty when placed in the body and is expanded with a therapeutic composition in situ activated elsewhere outside the body, and delivered through a passageway communicating with the chamber.

FIG. 4B is a cross-section view of two states, compressed state 41 and expanded state 42, of the same device 40, showing the pleats and folds 43 in the collapsed state 41, and the interior volume filled with activated therapeutic composition 44.

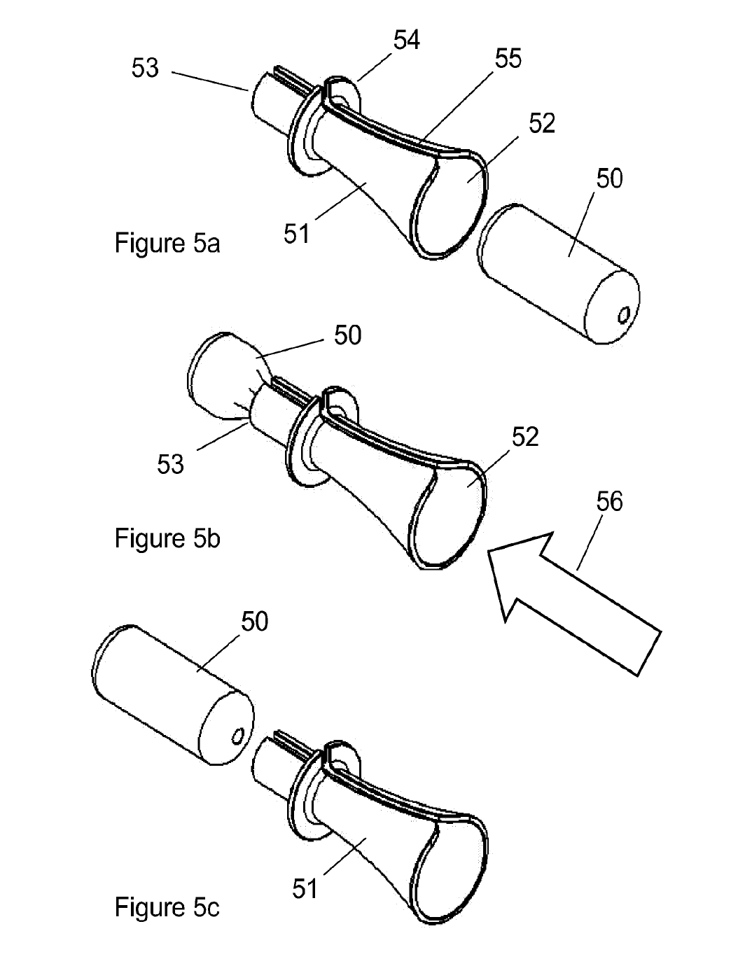

FIG. 5a shows an embodiment of the present invention device 50 that is designed to be delivered through a small opening in a body. In this embodiment, a flexible chamber houses a matrix that is elastically resilient, and springs back to roughly its original shape when de-compressed. A compression element 51 is included with a larger, first end opening 52 transitioning to a smaller second end opening 53. The smaller, second end opening 53 is placed in an opening in a body through which the device 50 is to be delivered. The compression element 51 may be equipped with a flange 54 to control its depth of insertion into the body opening, and a slot 55 to allow a continuous loop of suture passing through the device 50 to be removed through the side of the compression element 51.

FIG. 5b shows the embodiment device 50, after having been pushed into the larger first end opening 52 of compression element 51 in the direction of arrow 56, emerging from smaller, second end 53, as it would appear emerging into a body.

FIG. 5c shows device 50 having exited the smaller, second end opening 53 of compression element 51. Compression of the device 50 allows it to be implanted through an incision or body opening smaller than its own diameter. The elastic resiliency of the matrix contained within a flexible chamber allows the device to resume its shape inside the body. In an embodiment, the matrix contained within the chamber is activated by injection of BAA in situ.

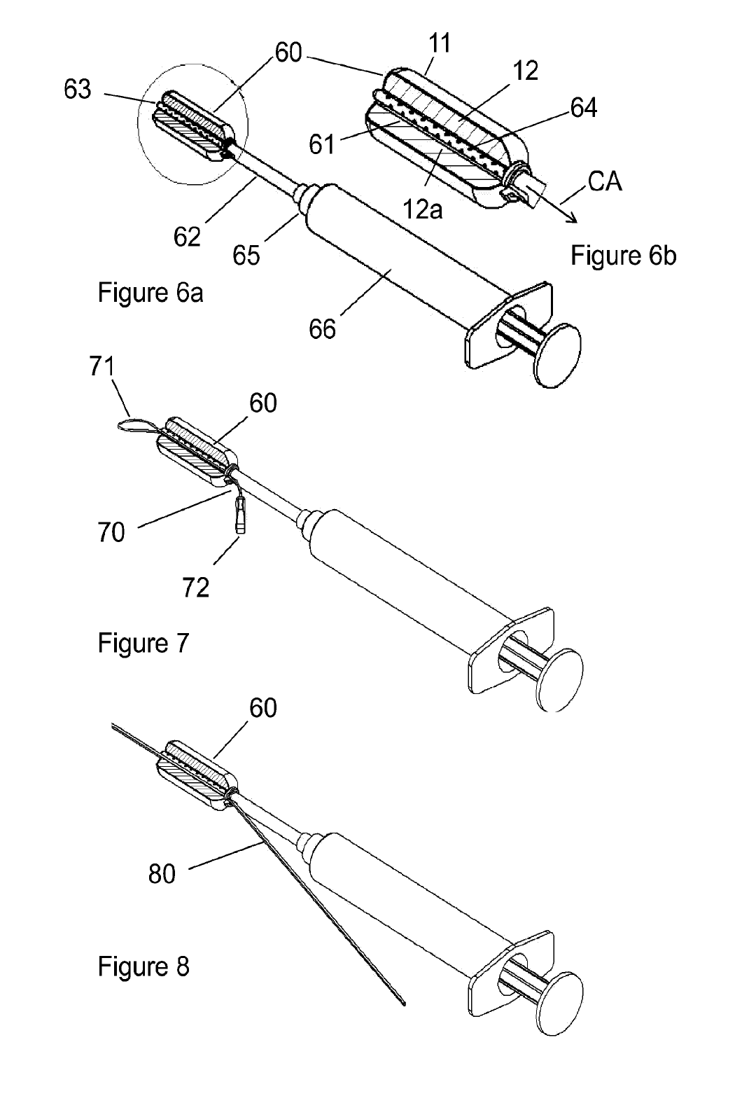

FIG. 6a and detail FIG. 6b show, in partial section view, an embodiment of the present invention device 60, having an outer element 11 defining an interior chamber filled with matrix 12, elongated along a chamber axis CA, and which interior chamber includes a cylindrical central void region 12a, shown in FIG. 6b as being filled with the removable passageway 62. As illustrated, the interior chamber 12 is shown with a matrix material therewithin. The device 60 includes a central opening 61 into which has been inserted a removable passageway 62 with a first end 63 running through the central opening 61 of the device 60 with a series of radial holes 64 distributed along the portion of its length running through the device 60, and second end 65 in fluid communication with a syringe 66 for injecting biologically active agent. Once activated, the fill tube 62 is removed.

FIG. 7 shows an embodiment of the present invention device 60 including a suture snare 70 with a first end formed in a loop 71 and a second end having a tab 72 to facilitate grasping with fingers. In practice, suture is threaded through the loop 71 and the user grasps the tab 72 and pulls suture through the central opening 61.

FIG. 8 shows an embodiment of the present invention device 60 where a suture 80 has been pulled through the central opening 61 shared with removable passageway 62. In an embodiment, suture 80 is used to guide device 60 into place in a body, or to secure it to tissue, or both.

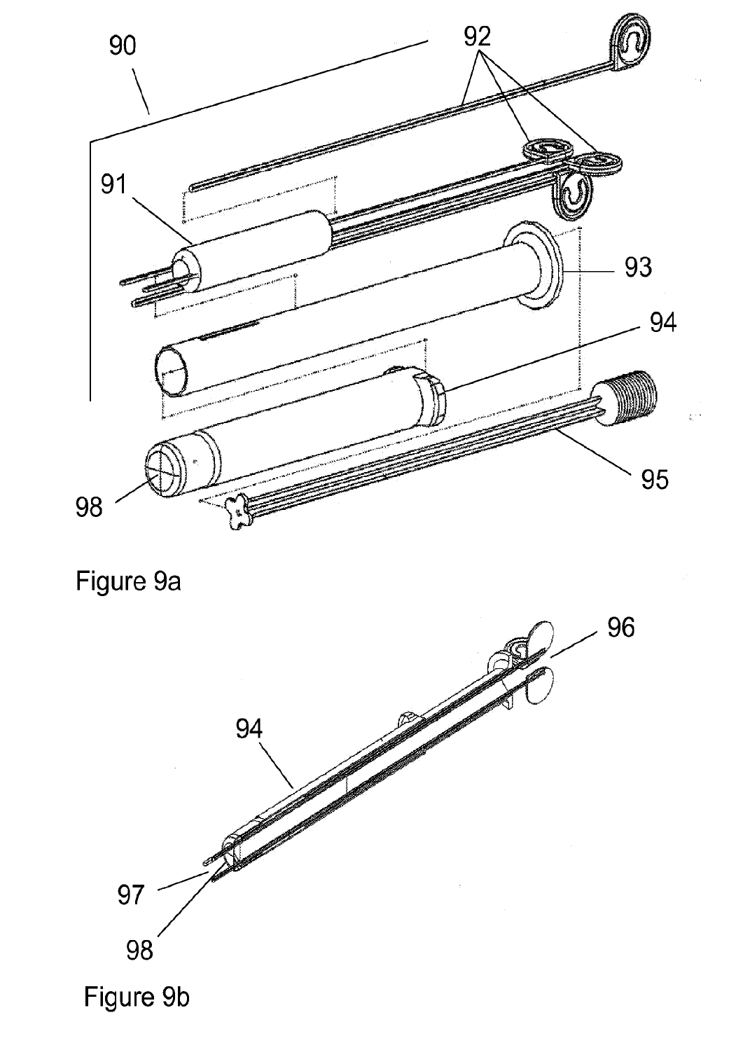

FIG. 9a shows an exploded view of another embodiment of the present invention. This embodiment comprises a matrix 91, one or more suture snares 92 inserted through the matrix, a passageway 93, a chamber 94 equipped with a split flexible opening 98, and a pusher 95.

FIG. 9b shows an isometric section view of the same device as shown in FIG. 9a, assembled for use. In use, guide or fixation sutures are pulled through the matrix using the suture snares. Biologically active agent is introduced into the proximal opening 96 of the passageway 93 (in an embodiment held vertically and dribbled in via gravity or, in another embodiment, injected from a syringe) where it activates the matrix to form an activated therapeutic composition. The entire assembly is slid along the guide/fixation sutures (not shown) into an opening in a body. Pusher 95, having slots to provide clearance for sutures, is introduced through the proximal end of the passageway 96 and pushes the activated therapeutic composition out of the distal end 97 of the chamber 94 that is equipped with a split flexible opening 98 to minimize backflow of body or irrigation fluids, yet allow the emergence of the therapeutic composition in the body.

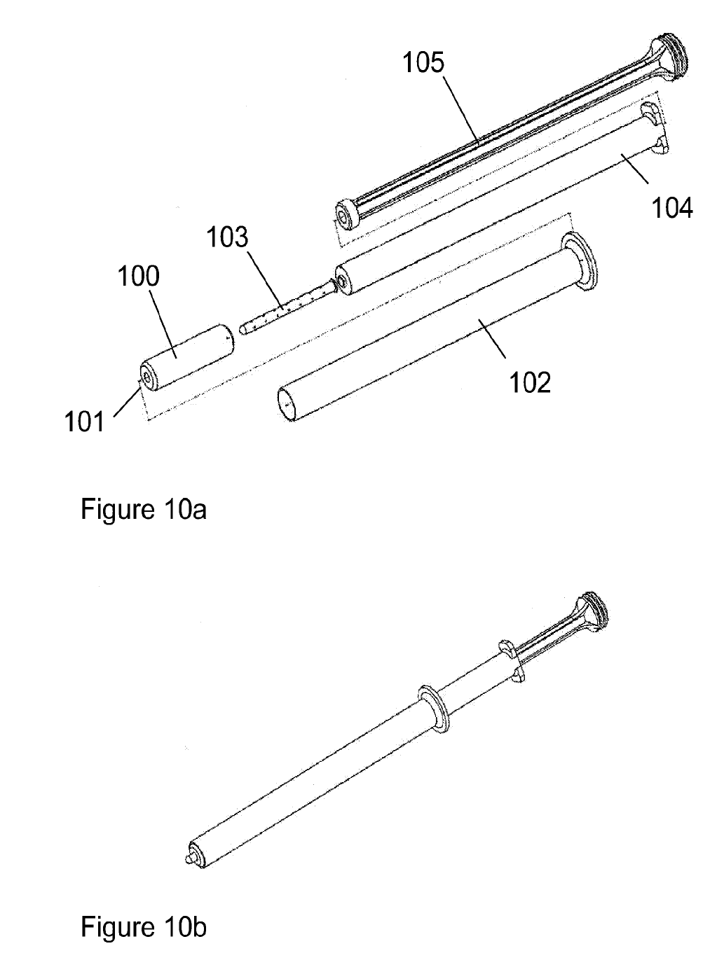

FIG. 10a shows an isometric exploded view of another embodiment of the present invention comprising a matrix 100 with a central opening 101, a chamber 102, a fenestrated passageway 103, and a means of propelling a quantity of biologically active agent through the passageway into the matrix in the chamber consisting of a cylindrical body 104 and a piston 105. FIG. 10b shows the same embodiment of FIG. 10a, in its assembled state.

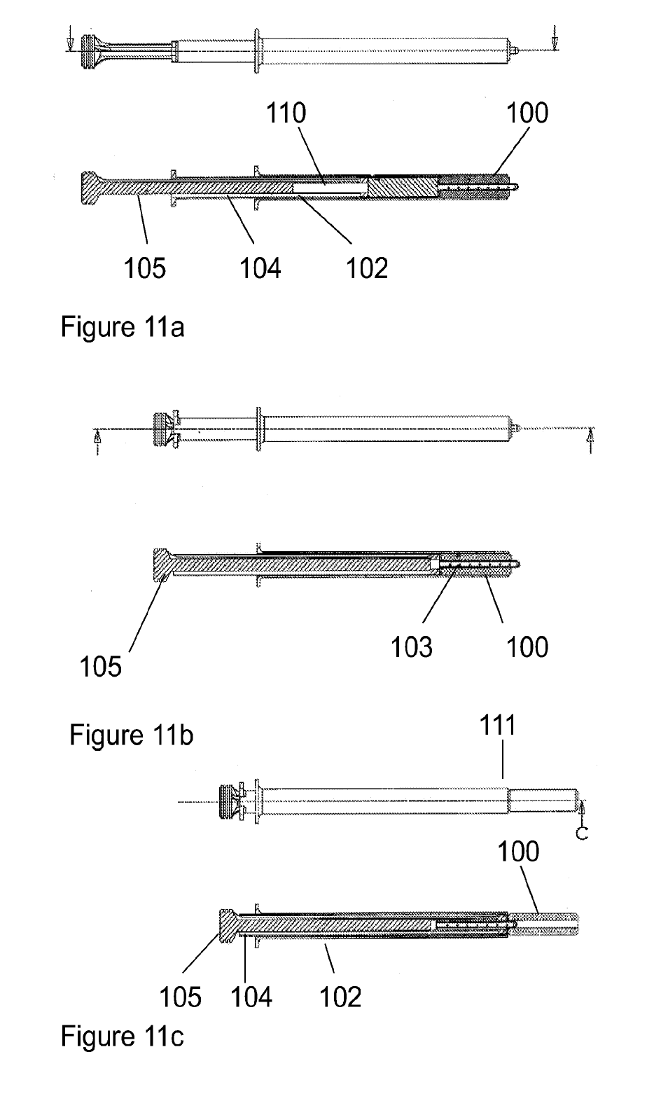

FIG. 11a shows this same device in plan view and section view as it would appear when ready for use. Chamber 102 contains a matrix 100. Cylindrical body 104 contains a quantity of biologically active agent (BAA) 110. Piston 105 and cylindrical body 104 translate linearly within chamber 102 and are both in a relatively retracted position.

FIG. 11b shows piston 105 translated to the right, displacing biologically active agent through fenestrated passageway 103 and into matrix 100, thereby forming an activated therapeutic composition.

FIG. 11c, shows the device the distal end 111 of the chamber 102, has been place in a body (not shown). Cylindrical body 104 is translated to the right, ejecting matrix 100, which has now become an activated therapeutic composition, into the body adjacent a tissue to be treated.

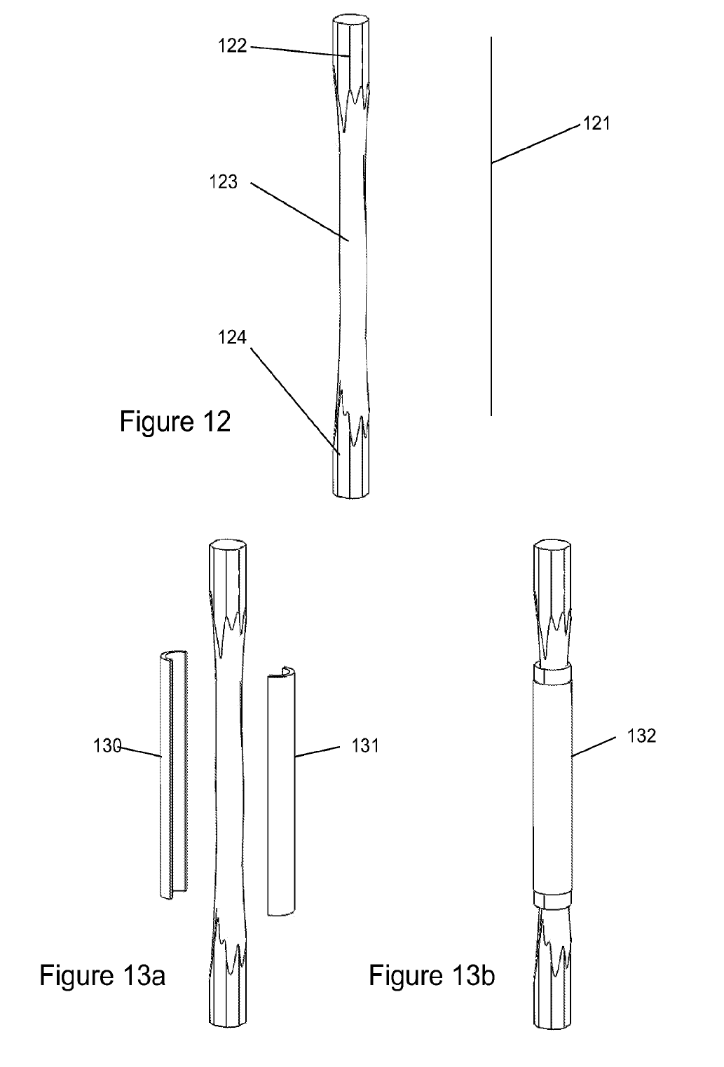

FIG. 12 shows a bone-tendon-bone autograft 121 typical of the type used in best practice ACL reconstruction surgery at the time of this writing. The graft comprises a proximal end 122 of bone harvested from the patient's patella, a graft midsection tendon portion 123 harvested from the patient's patellar ligament, and a distal end 124 of bone harvested from the patient's tibia. The proximal and distal end boney plugs have been trimmed to pass freely through an 8 mm bone tunnel. Although a bone-tendon-bone autograft will be used throughout this description the same invention application applies to all types of tissue grafts, including but not limited to soft tissue (e.g. hamstring) autografts, and all types of allografts and xenografts used for connective tissue reconstruction.

FIG. 13a shows a preferred embodiment for applying a therapeutic composition to a graft extra-corporeally, prior to implantation. This example shows two halves of a split tube 130 and 131 of matrix material. Other embodiments employ a tubular matrix with one split side that can be spread open to wrap around the graft. Still other embodiments use a graft split into three or more segments that are placed around the graft. In an embodiment the matrix is a sponge of collagen, such as Type I collagen. FIG. 13b shows the saturated collagen tube halves placed around the tendon portion of the graft and held in place by a chamber 132. Once so assembled, biologically active agent is applied to the exposed end of the matrix where it wicks along the entire length of the matrix. To accelerate the wicking process, a passageway like a small tube may be inserted into the space in the chamber occupied by the matrix and biologically active agent may be forced in to facilitate wetting and activation of the matrix. The cross sectional diameter of the treated graft remains less than the bone tunnel diameter (e.g., 8 mm) and therefore able to slide freely into place where it will be fixed in place using interference screws, cross pins or other fixation means known to the art.

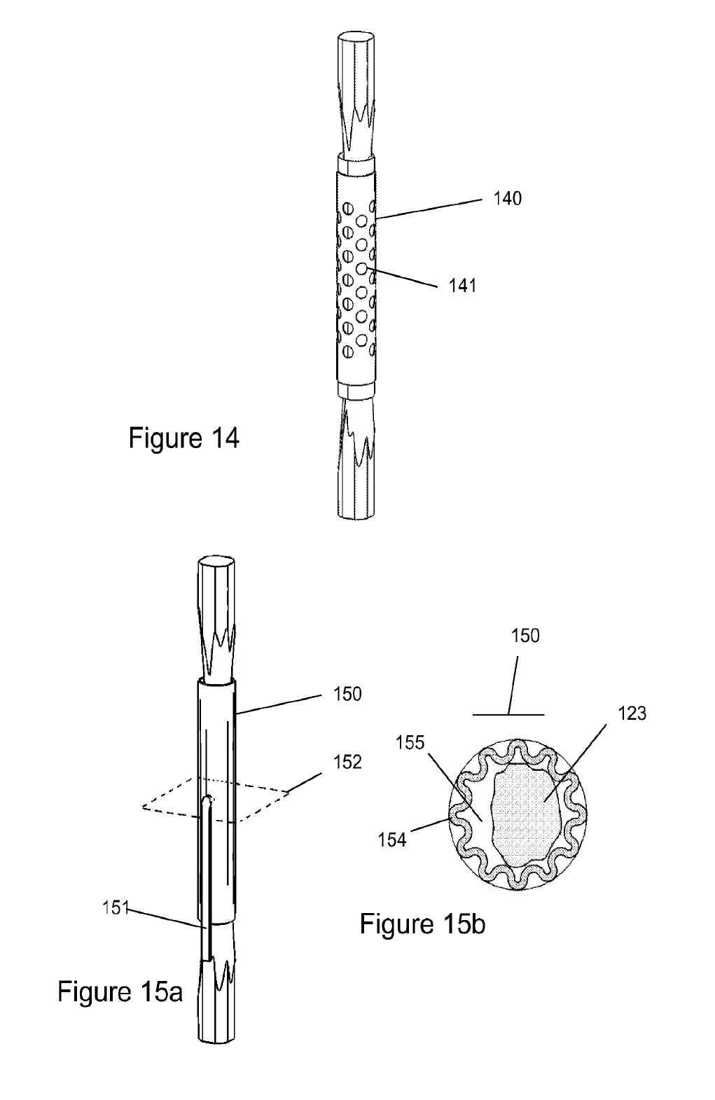

FIG. 14 shows a variation of the previous embodiment where the chamber 140 is provided with a series of passageway holes 141 thereby allowing the graft to be placed and fixated dry, and to have activating solution applied to the exterior of the prepared graft in situ.

FIG. 15a shows an example of a class of embodiments where a chamber containing a compressed matrix is fitted around the graft extracorporeally and filled with flowable biologically active agent in situ. A pleated chamber 150 with a fill-tube 151 is slid in place over the graft. FIG. 15b shows a section of the embodiment at plane 152, showing a series of pleated folds 154 around its circumference surrounding graft mid-section 123 and space filled with compressed matrix.

FIG. 16 shows this same embodiment as it would appear in situ (joint and fixation means not shown) during injection of BAA. The intra-articular portion 160 of the chamber 150 expands as flowable biologically active agent is injected via syringe 161, thereby expanding the compressed matrix and creating a large mass of therapeutic composition to provide increased cell growth and strength to the graft. In another embodiment the chamber is fitted around the graft and installed in the joint empty, and a flowable therapeutic composition, activated extracorporeally, is injected into the chamber.

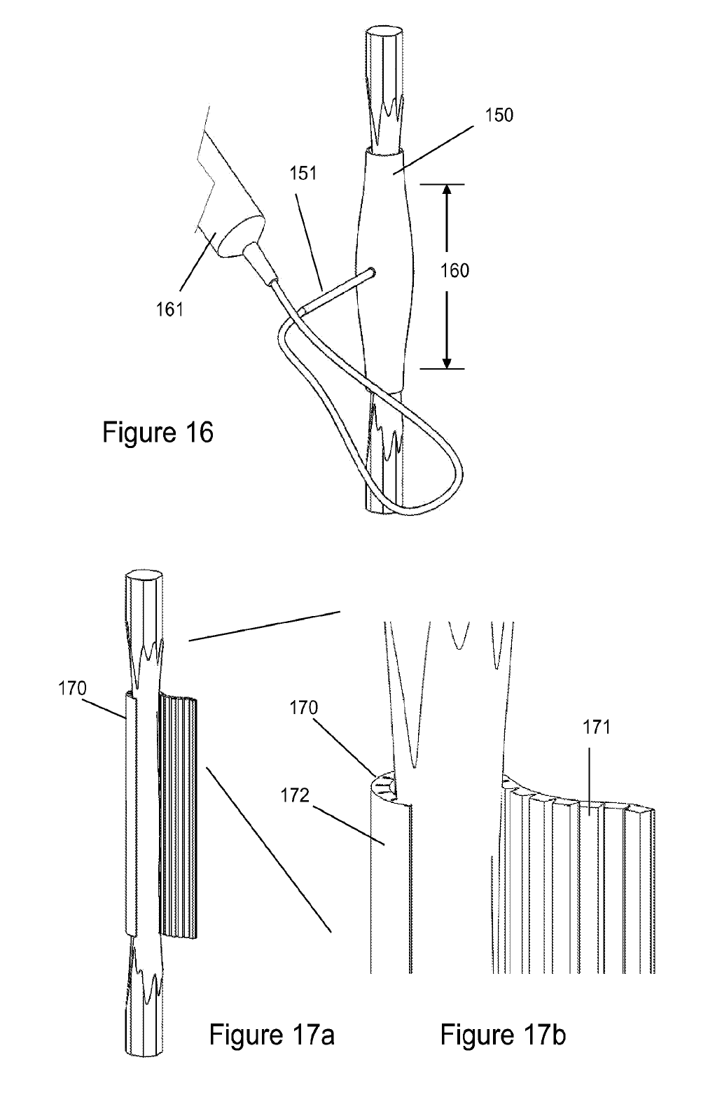

FIG. 17a shows an example of a class of embodiments including a wrap-around matrix with a barrier coating. When closed around the graft, the barrier sheet forms a chamber containing a matrix. FIG. 17a shows a graft partially wrapped in the wrap-around collagen sheet with a barrier coating 170. FIG. 17b is a detail view of FIG. 17a showing individual collagen strips 171 held together by a barrier coating on the outside 172 of the sheet. The sheet is tied around the graft with sutures or other fasteners and the matrix is activated in any of the previously disclosed means (not shown). The activated wrap-around chamber can be used extracorporeally prior to graft implantation, or placed intracorporeally provided it is of sufficient length to fit inside the articular space.

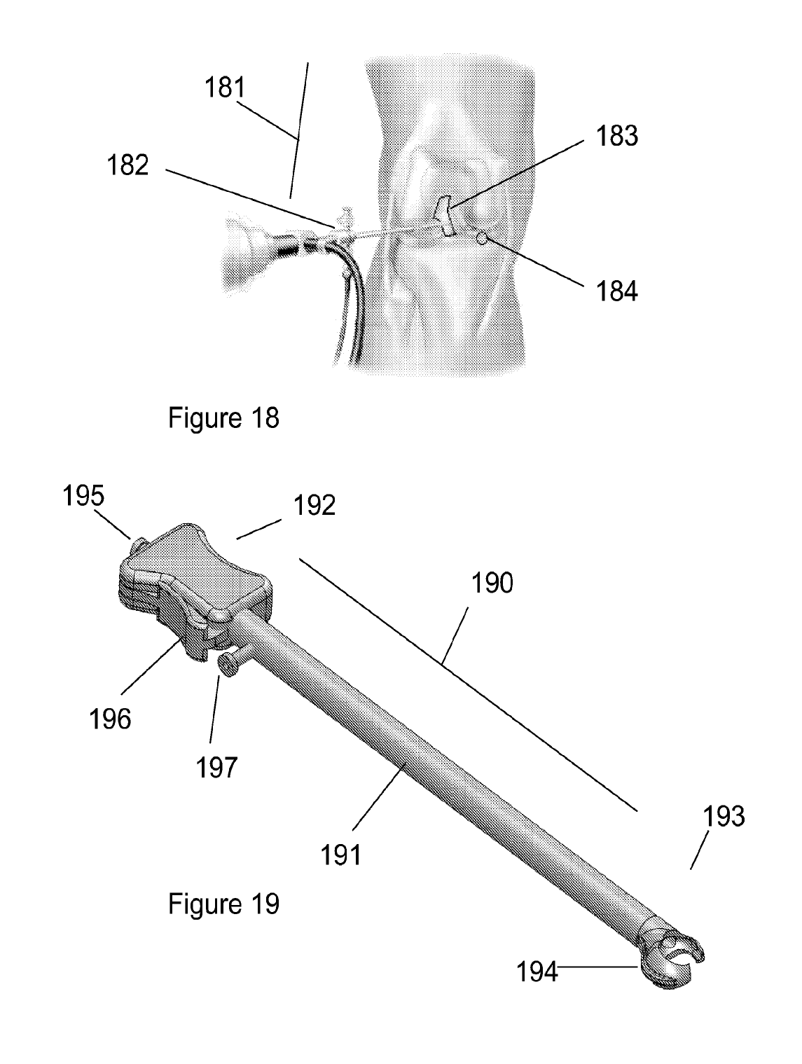

FIG. 18 shows a typical arrangement of a knee 181 for arthroscopic surgery. An arthroscope 182 enters the joint through an anteriolateral portal or incision. An anterior cruciate ligament (ACL) 183 to be treated with a therapeutic composition can be accessed through an anteriomedial portal 184.

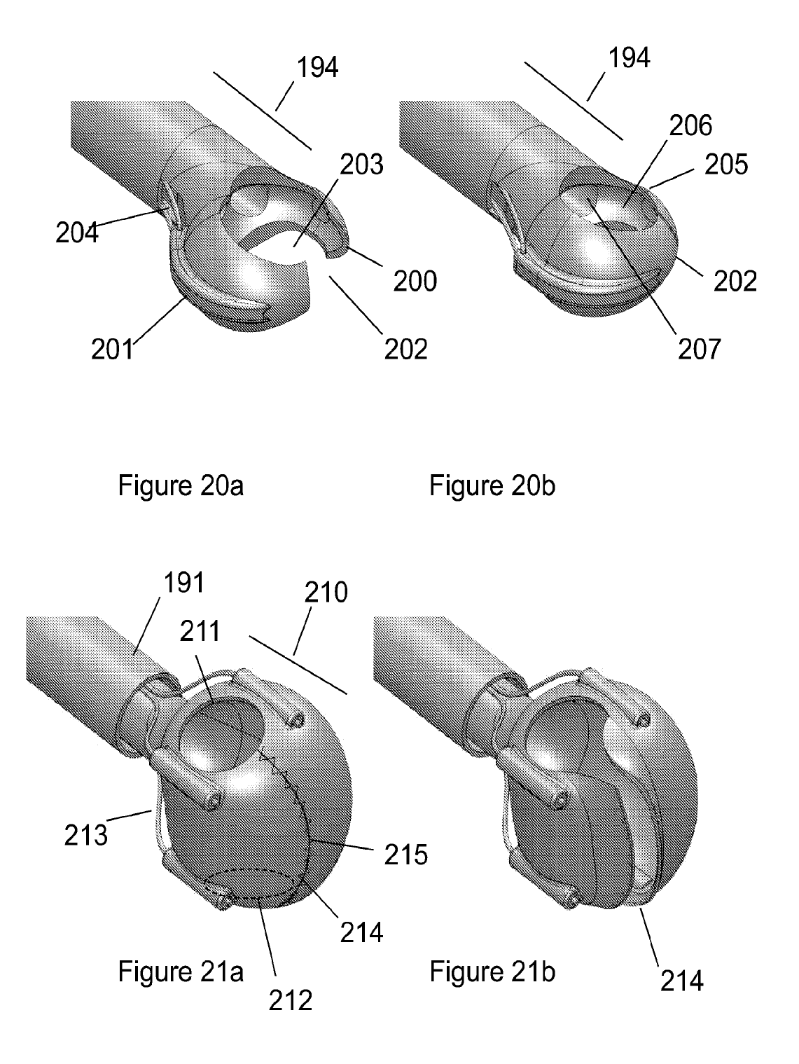

FIG. 19 shows an embodiment of the present invention 190 comprising a hollow shaft 191 and terminating in a proximal end 192 and a distal end 193. Distal end 193 includes a containment chamber 194. Proximal end 192 includes a connector means 195 in communication with the lumen of hollow shaft 191 for attachment to a therapeutic composition preparation and advancing means, not shown. In other embodiments the therapeutic composition preparation and advancing means is integral to the device. In some embodiments, proximal end 192 further includes a control button 196 connected by a linkage (204 in FIG. 20a) for articulation of the containment chamber 194. In some embodiments proximal end 192 further includes a connector means 197 for connection to a gas supply (not shown). Gas supply connector 197 communicated directly with containment chamber 194 through a secondary passage (not shown) running through shaft 191.

FIG. 20a shows an embodiment of a containment chamber 194 at the distal end of the device. In this embodiment the entire containment chamber is molded in a flexible elastomer such as silicone rubber. The containment chamber 194 includes a fixed portion 200, and an articulating portion 201 separated by a gap 202. A linkage 204 connects to a control button (196 in FIG. 19) at the proximal end of the device. When actuated, the control button moves linkage 204 distally to flex articulating portion 201 as shown in FIG. 20b, where gap 202 has closed completely. In practice, when used to treat an ACL with a therapeutic composition, the distal end 193 of device 190 is inserted into anteriomedial portal 184 of knee 181. Containment chamber 194 is positioned with ACL 183 (not shown in FIG. 20a) in gap 202. Control button 196 is actuated to close articulating portion 201 around ACL 183. Top and bottom aligned openings 205 fit snugly around the ACL 183 leaving toroidal region 206 isolated from the surrounding environment of the knee joint which is filled with circulating saline fluid. At this point, therapeutic composition preparation and advancing means (not shown) can advance malleable composition through hollow shaft lumen 207 and into the toroidal region 206 surrounding the ACL, displacing any fluid that may be trapped in the toroidal region 206. Alternatively, a gas such as air or carbon dioxide may be introduced into the toroidal region 206 to displace fluid prior to introduction of the therapeutic composition. Formulations of therapeutic composition that transition from a non-cohesive state to a cohesive state, where the cohesive state is impervious to saline, but the non-cohesive state is not, will requite the chamber to remain closed around the ACL until the transition is complete, at which time the control button is again activated to open the articulating portion 201, releasing the ACL and leaving a cohesive mass of therapeutic composition encircling the full diameter of the ACL.

FIG. 21a shows another embodiment of the present invention that has particular utility for the treatment of grafts used to reconstruct a torn ACL in the knee. The ACL reconstruction procedure involves drilling bone tunnels through the tibia and femur, passing a graft (allograft, autograft, or xenograft) through the tunnels, and fixing the tibial and femoral ends in place. In this embodiment the graft is passed through aligned openings 211 and 212 in containment chamber 210 prior to fixation of one or both ends. In practice, containment chamber 210, being made of an elastomeric material like silicone, is collapsed and housed inside hollow shaft 191, allowing it to be inserted into the joint through an anteriomedial portal. Once inside the joint the containment chamber 210 is ejected from the distal end of the hollow shaft 191 where it expands to its unconstrained shape as shown. To facilitate ejection and expansion, some embodiments include a network of spring wires 213. The containment chamber 210 is next advanced into the intracondylar notch of the femur where openings 211 and 212 are aligned with previously drilled femoral and tibial bone tunnels respectively. The graft is passed through the tunnels such that the proximal end of the graft resides in the femoral tunnel and the distal end of the graft resides in the tibial tunnel, and the mid-section of the graft passes through the containment chamber 210 in the condylar notch. The surgeon then fixes the proximal and distal ends of the graft to the femur and tibia respectively. Like the embodiment in FIG. 20, therapeutic composition is then advanced into the containment chamber directly, displacing ambient fluid as it fills the structure, or in other embodiments, gas is introduced to displace fluid prior to advancing composition. Once therapeutic composition has fully enveloped the mid section of the graft, and has transitioned from a non-cohesive state to a cohesive state, the containment structure may be removed. In an embodiment a seam 214 on the distal aspect of the containment chamber extending from opening 211 to opening 212 is held closed by a temporary closure, in this embodiment, a sewing stitch 215 that releases when one end of the thread is pulled. Other embodiments use other temporary closure means such as zippers, molded-in wires that tear the wall when pulled, thin wall sections or weak glue seams that fail under a predetermined load, or any of many other release mechanisms known to the art. FIG. 21b shows the temporary closure released and seam 214 open, allowing the containment chamber to be pulled off the mass of cohesive therapeutic composition enveloping the graft. The containment structure 210 is then pulled back into the hollow shaft 191 and the device is removed from the joint. The therapeutic composition remains to treat the graft.

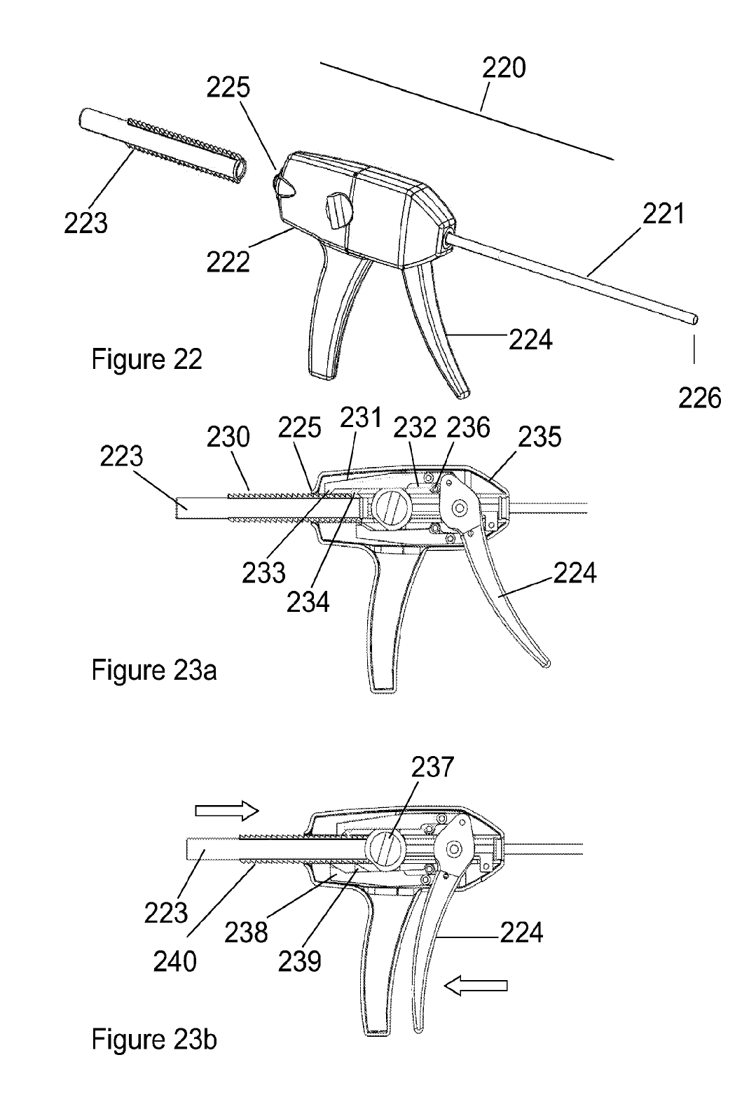

FIG. 22 shows an embodiment of the present invention 220, comprising a hollow shaft chamber 221, a handle 222, a reservoir 223, and a trigger lever 224. The device has a proximal 225 and a distal 226 end. Although the distal end 226 is shown as being cylindrical, in the alternative, the distal end 226 may include a modified tip that would allow the composition to be extruded as a ribbon, an oval, or other polygonal configuration in cross-section.

FIGS. 23a and 23b show this same embodiment of the device with one half of the handle housing removed to reveal the operating mechanism. In FIG. 23A the reservoir 223 having integral ratchet teeth 230 has been inserted into the proximal end 225 of the device where it engages with a drive mechanism comprising a pivoting and translating advancement arm 231 and a pivoting advancement ratchet pawl 232. The advancement arm 231 includes a hook 233 engaging the reservoir ratchet teeth 230 and is attached to trigger lever 224. The advancement ratchet pawl 232 also has a hook 234 engaging the reservoir ratchet teeth 230 and is attached to the handle housing 235 at a pivot 236.

FIG. 23B shows the reservoir 223 advanced distally by means of squeezing the trigger lever 224, causing the advancement arm 231 to translate in a distal direction, pulling the reservoir 223 with it. The pawl hook 234 clicks over the advancing reservoir ratchet teeth 230 until the trigger lever 224 reaches its maximum travel. The advancement pawl 232 then engages the reservoir ratchet teeth 230 preventing return motion in the proximal direction while the user relaxes her grip, allowing a return spring (not shown) to move the trigger lever 224 to its starting position, causing the advancement arm 231 to translate in the distal direction. At the end of its translation the advancement arm hook 233 re-engages the reservoir ratchet teeth 230 and the process is repeated. In this way, repeated squeezing of the trigger lever 224 causes the reservoir 223 to translate its full length in the distal direction.

A cam 237 is provided such that when activated (turned 45° counter clockwise) it disengages the advancement arm 231 and the advancement ratchet pawl 232 and engages a retraction arm 238 and retraction pawl 239 that engage a set of retrograde ratchet teeth 240 on the reservoir 223. In so doing, the mechanism is reversed and translation of the reservoir occurs proximally, allowing the device to draw fluids into the reservoir or release the reservoir from the device.

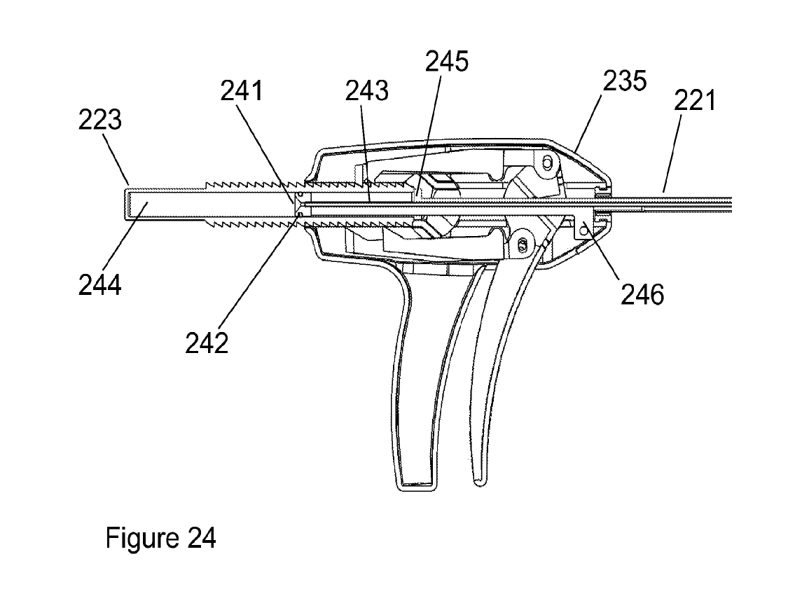

FIG. 24 shows a section view of the same embodiment of the device showing the internal components that affect the activation of the matrix to form a therapeutic composition and is ejection from the device. In this view we see a plunger 241 with a seal 242 attached to a hollow needle passageway 243. The plunger, seal and needle passageway are attached to the handle housing 235 and do not translate. The reservoir 223 is shown in cross section revealing a fluid storage region 244, filled with liquid biologically active agent, in communication with the inner lumen of the needle passageway 243. As the reservoir is advanced in the distal direction by the advancement mechanism, fluid in the fluid storage region 244 is forced through the needle passageway. Also, as the reservoir 223 advances, it pushes against a pusher tube 245 that translates distally around the needle and through the shaft chamber 221. The proximal portion of the pusher tube 245 has a slot that allows it to slide over a tang 246 that supports the needle and attaches the needle/plunger assembly to the handle housing 235.

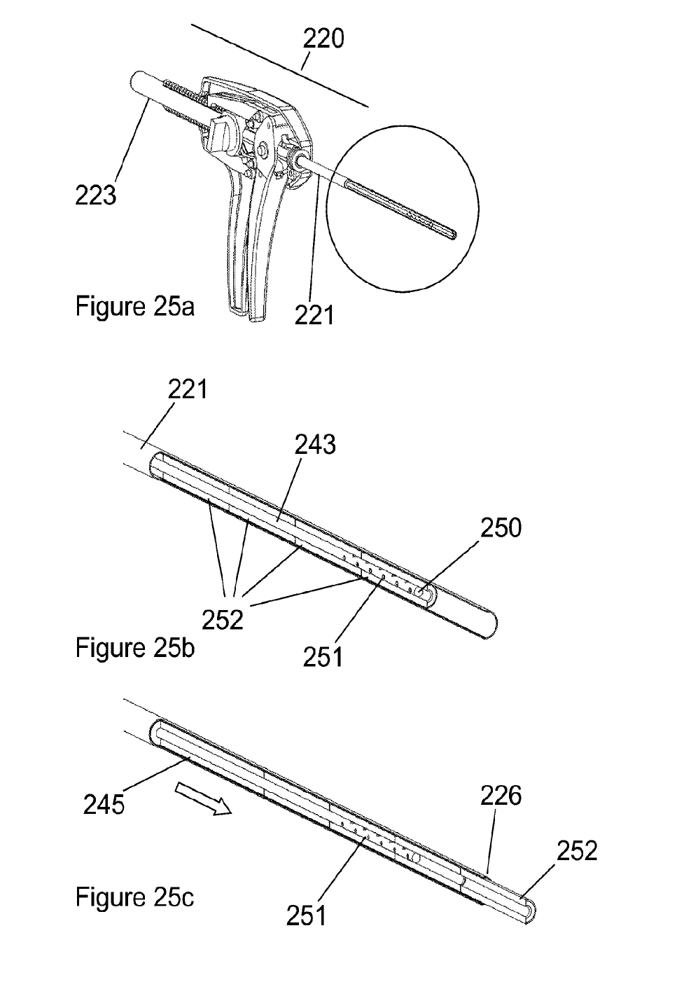

FIGS. 25a, 25b and 25c show the distal portion of the same embodiment of the device shaft chamber 221 in cross section. FIG. 25a shows the orientation of the device and the portion of the device (circled) detailed in FIGS. 25b and 25c.

FIG. 25b shows the hollow needle passageway 243 welded closed at its distal end 250 with a distributed series of transverse holes 251 near the distal end communicating with the inner lumen. Filling the space between the inner lumen of the shaft chamber 221 and the outer wall of the hollow needle passageway 243 are a series of tubular matrix segments 252.

FIG. 25c shows the pusher tube 245 advancing distally, moving the tubular matrix segments 252 distally and eventually ejecting them from the distal end 16 of the device. Simultaneously to movement of the matrix segments, a biologically active agent is pumped from the reservoir, through the hollow needle passageway 243, out the distributed series of transverse holes 251, and into the pellets 252, wetting them with a biologically active agent and thereby creating an activated therapeutic composition as it is being ejected from the device. The inside diameter of the reservoir 223 is designed such that 1 cm of translation of the reservoir inject the exact amount of a biologically active agent needed to activate 1 cm of matrix segment, thereby assuring the ideal desired ratio of matrix and a biologically active agent.

Those skilled in the art will recognize that many other embodiments can be conceived that will achieve the same end function. Other embodiments substitute the ratcheting drive mechanism with a screw mechanism, hydraulic or pneumatic actuator, direct acting syringe-type plunger, motor drive, peristaltic pump, or any of the variety of means known for initiating movement of fluids and solids. Still other embodiments achieve ideal ratio of therapeutic composition components through the use of other than 1:1 drive means and instead use a plurality of drive means with different proportional rates of advancement. Still other embodiments provide for other combinations of components with different consistencies such as pastes, gels, foams, dry powders, liquids, etc. in any combination and number of components.

In another special embodiment of the invention, where the biologically active agent component of the therapeutic composition is autologously derived PRP, the additional means of processing whole blood to extract PRP is included as part of the invention.

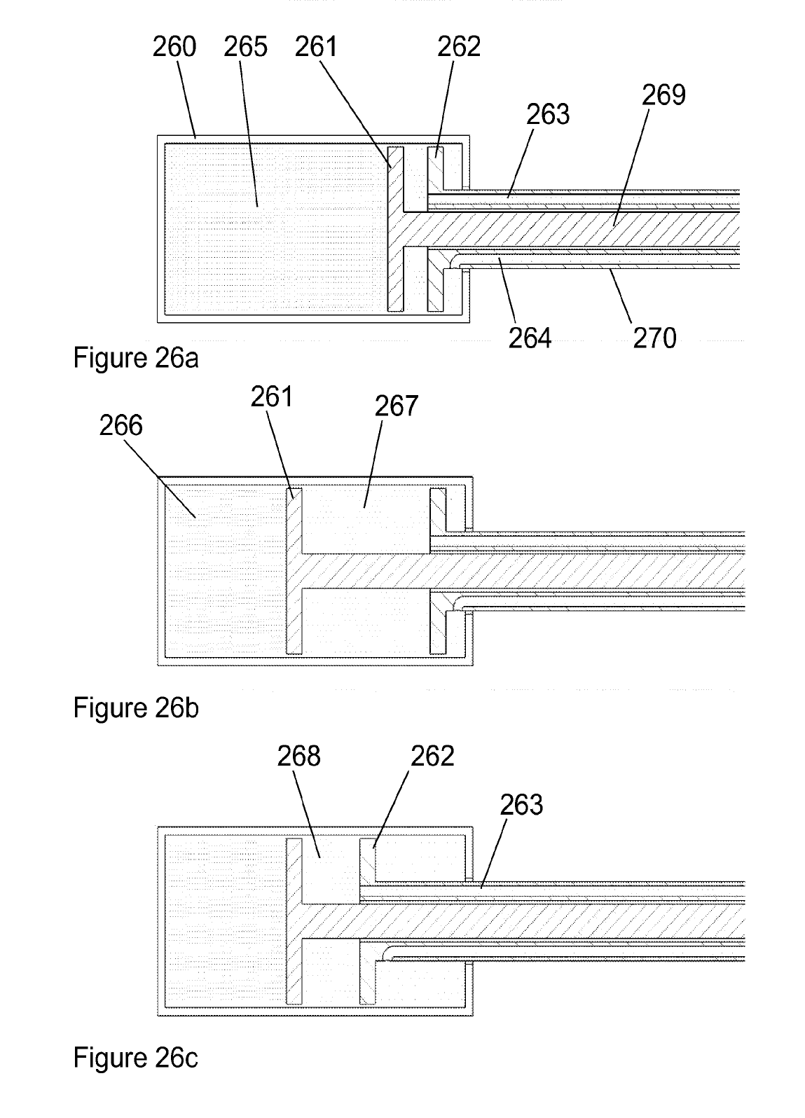

FIGS. 26a, 26b and 26c are a schematic representation of one embodiment of an integral PRP extraction system incorporated into the device. FIG. 26a shows a cross section of a schematic representation of a filter-based PRP extraction system. In an embodiment, cylindrical reservoir 260 takes the place of reservoir 223 in FIG. 1. In one embodiment reservoir 260 is fixed relative to the handle 222, and a first plunger with a first filter membrane 261, and a second plunger with a second filter membrane 262, move independently, each being driven by a separate trigger lever and reversing ratchet and pawl system (not shown, but like that comprised of items 224, 231, 232, 237, 238 and 239 shown previously), acting on first and second plunger/filter shafts 269 and 270. A first passageway 263 communicate with the space between first plunger 261 and second plunger 262. A second passageway 264 communicates with the space between the second plunger 262 and the right side wall of the reservoir 260. The left side of the reservoir is filled with whole blood 265.

FIG. 26b shows first plunger with a filter membrane 261 moved to the left. The first filter membrane has a pore size sufficiently large to permit passage of platelets through the membrane but small enough to inhibit the passage of most red and white blood cells. The result of this filtering step is a region of concentrated red and white cells 266 and a region of plasma and platelets with 1× the concentration of platelets in whole blood 267.

FIG. 26c shows the second plunger with a second filter membrane 262 moved to the left. The second filter membrane has a pore size sufficient to prevent the passage of platelets while still allowing the passage of plasma. The result is a region with a volume of platelet rich plasma (PRP) 268 which may be extracted through first passageway 263 for use as the biologically active component of a therapeutic composition. Other embodiments eliminate the second plunger and second filter membrane 262 and use 1×PRP as the biologically active component of a therapeutic composition. The means of driving the plungers and extracting the PRP may be any of the mechanism means described above. In alternative embodiments, saline or other biologically neutral or inactive components may be used instead of or in addition to PRP.

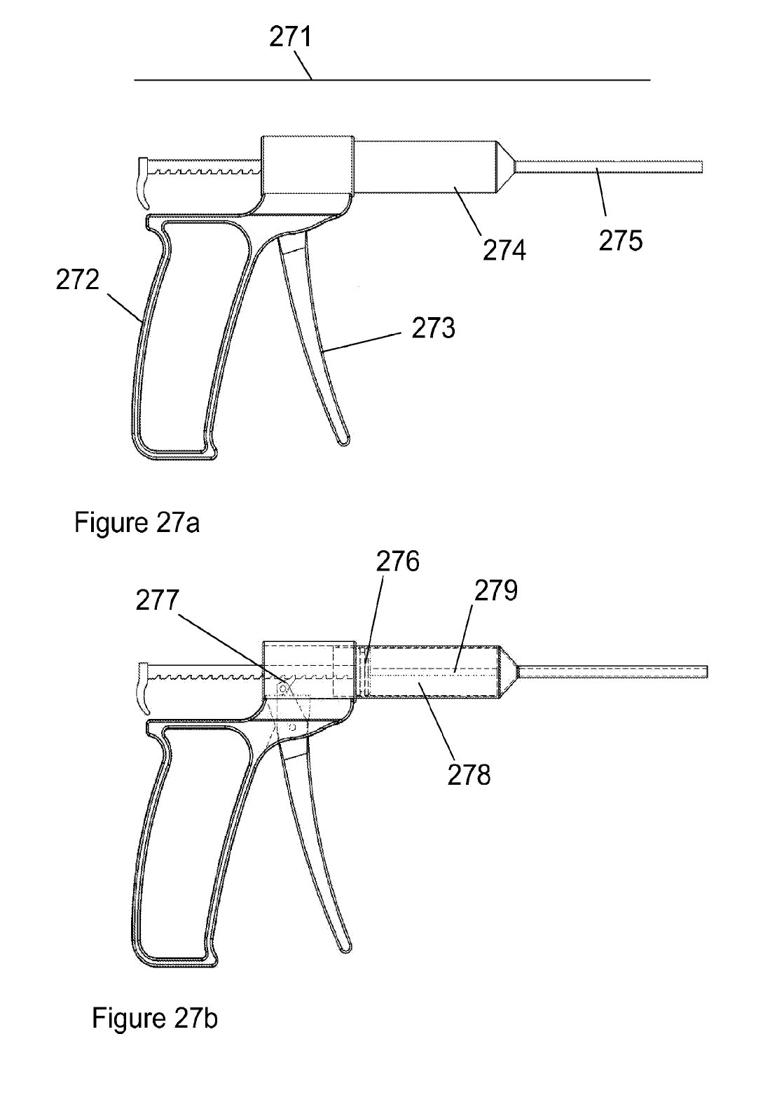

FIG. 27a shows an embodiment of a therapeutic compound dispensing element 271 of the present invention. The element 271 comprises a handle 272, a trigger 273, a syringe-like dispensing chamber 274, and a delivery channel 275.

FIG. 27b shows the interior parts of the same element 271, including a ratcheting mechanism 277 for advancing a piston 276 inside the chamber, and a sponge-like matrix 278 equipped with a central opening 279.

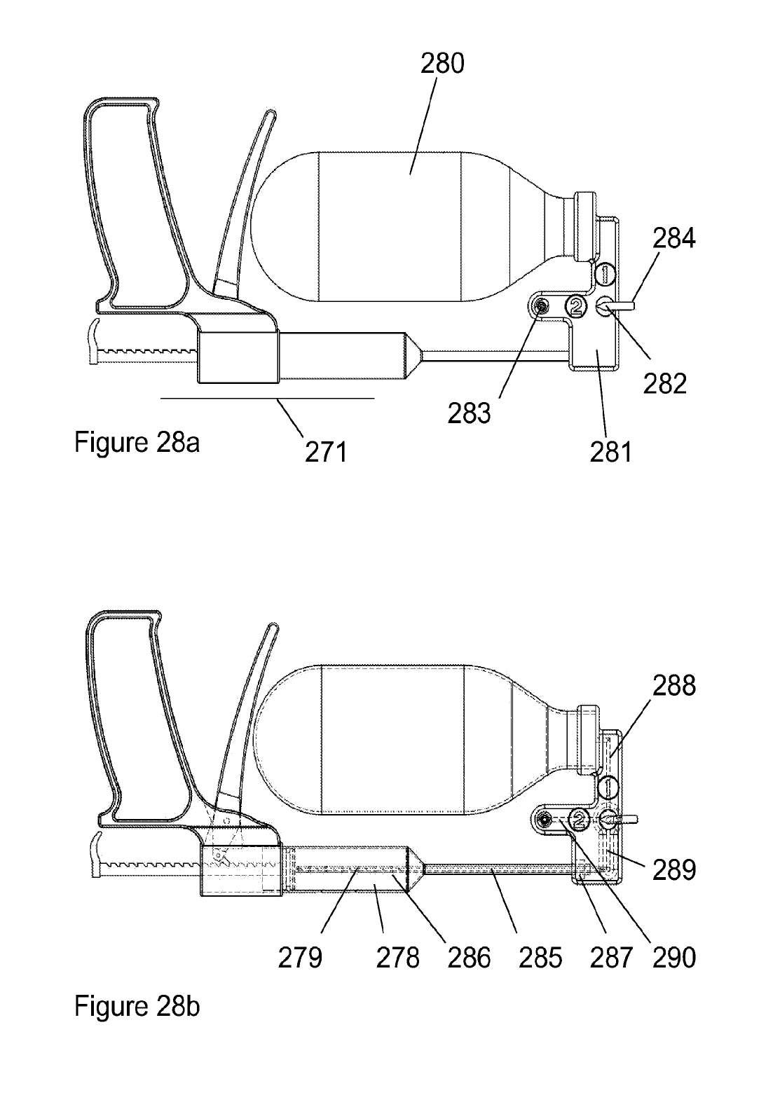

FIG. 28a shows an embodiment of the assembled invention, comprising the dispensing means 271, a vacuum supply 280, a valve manifold 281, a switching valve 282 with a handle/direction indicator 284, and a connector 283 for connecting to a reservoir of a liquid biologically active agent (not shown).

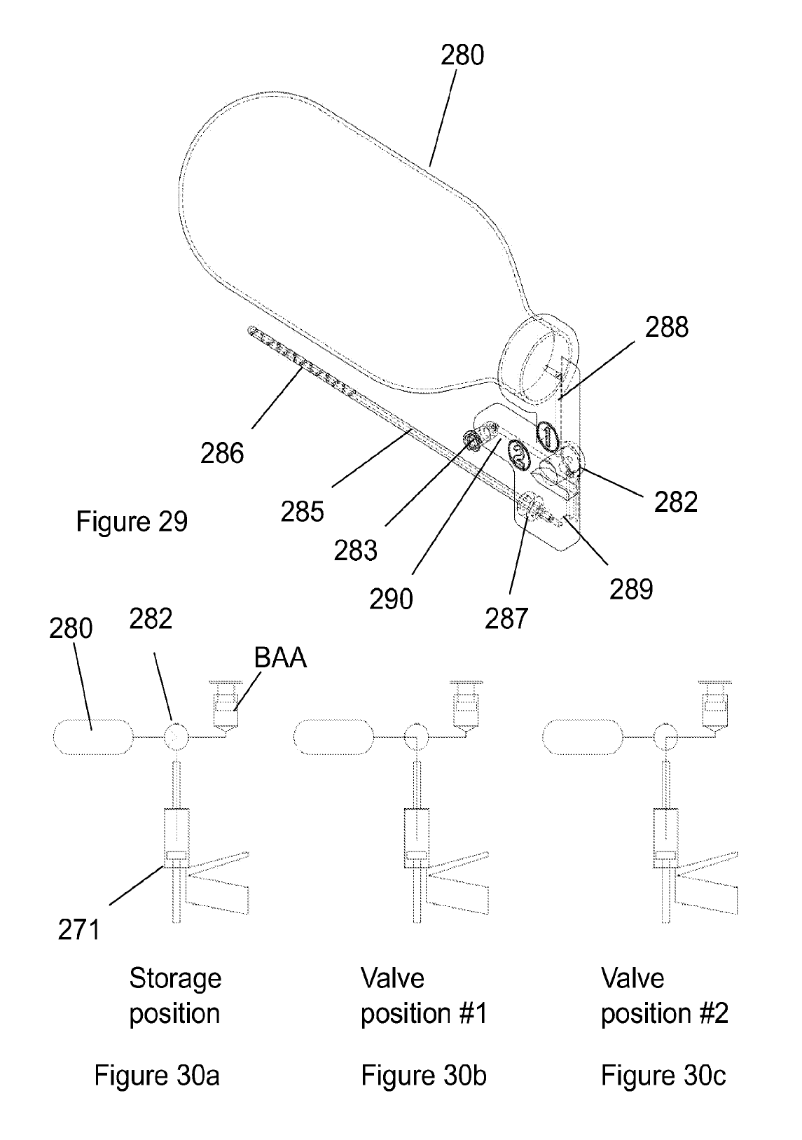

FIG. 28b shows interior components of the same invention, including passageway 285 passing through delivery channel 275 of dispensing component 271, and into the central opening 279 of sponge-like matrix 278. The portion of passageway 285 residing in central opening 279 is equipped with distributed radial holes 286 to uniformly distribute a liquid biologically active agent over the length of the sponge-like matrix. A leak-tight seal 287 engages the exterior of the delivery channel and the valve manifold to create an enclosed system. As shown in FIG. 29, vacuum supply 280 communicates with switching valve 282 by means of a conduit 288. The interior lumen of hollow tube passageway 285 communicates with switching valve 282 by means a conduit 289. A reservoir of a liquid biologically active agent (not shown) connects to connector fitting 283 and communicates with switching valve 282 by means of a conduit 290.

In operation, an operator, usually the operating room scrub nurse, will open a sterile package containing the invention as shown in FIG. 28a. The operator will connect a reservoir of, for example, liquid biologically active agent, or a syringe of autologous platelet rich plasma, or other biologically active or biologically neutral agents, to connector fitting 283. The operator will then turn the handle/indicator 284 of switching valve 282 from its right-pointing “3-o'clock” storage position (shown in FIG. 30a) counter clockwise to the “12-o'clock” position “1” (shown in FIG. 30b), and in so doing connect vacuum supply 280 to chamber 274 containing sponge-like matrix 278 through conduits 288, 289, passageway tube 285, and radial holes 286. This will equalize the pressures of the vacuum supply and the sponge-like matrix 278. The operator then turns the valve handle/indicator CCW to the “9-o'clock” position “2” (shown in FIG. 30c). This action will first re-close conduit 288 to the vacuum supply, and then open communication between the reservoir of liquid, biologically active agent connected to connector fitting 283 to the sponge-like matrix 278 by means of passageways 290, 2894 passageway tube 285 and radial holes 286. In doing so, the sponge-like matrix will become fully saturated in the absence of air. Piston 276 is advanced until pressure in chamber 274 is equalized with surrounding ambient pressure, compacting and concentrating the now activated therapeutic composition. The user then grasps the dispensing component 271, pulling the delivery channel 275 out of the seal 287 into the valve manifold 281 and hands the dispensing component containing the activated therapeutic composition to the surgeon who delivers the composition to the patient's body.

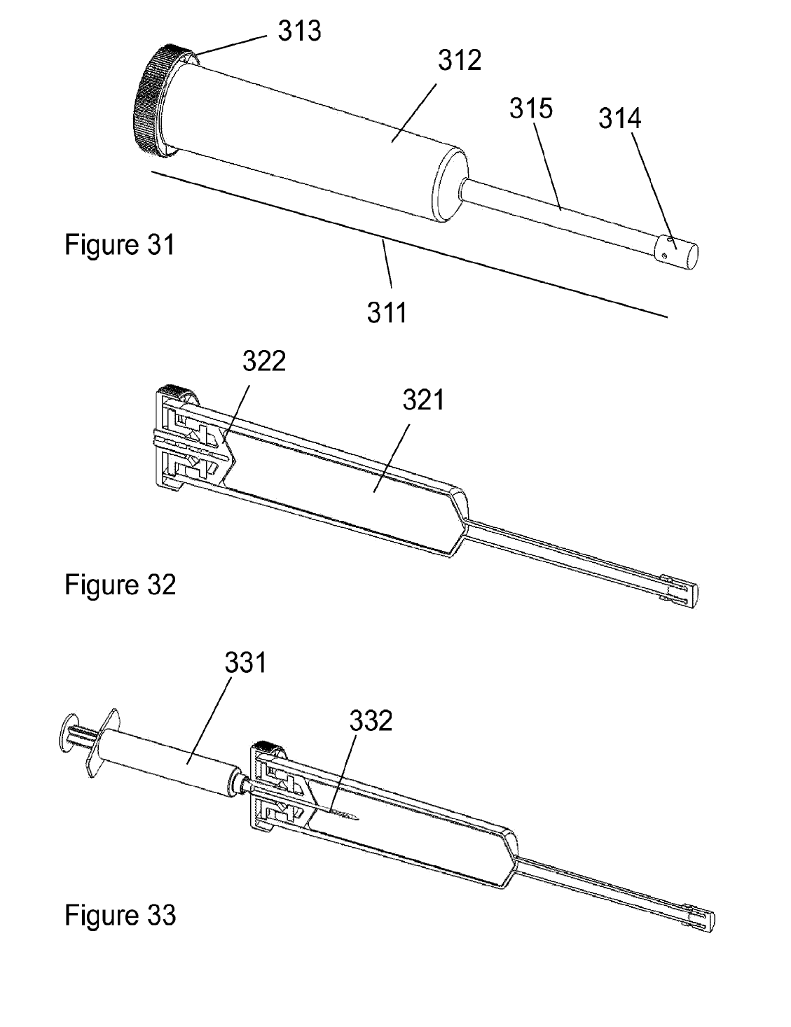

In an embodiment (shown) the vacuum supply 280 is an enclosed flask evacuated during manufacture of the device. In other embodiments the vacuum source may be a vacuum pump (manual or motorized), hospital wall suction, or simply a manually back-drawn syringe, or any other negative pressure sources known to the art. In an embodiment (shown) the switching valve 282 may be a cross-drilled stopcock. In other embodiments any of the multitude of multi-way valve types known to the art may be employed. In still other embodiments multiple single-way valves may be used to achieve the same effect. Still other embodiments employ different styles of dispensing means, tubes, advancing mechanisms, etc. all known to the art but achieving the same end result. FIG. 31 shows another embodiment of the invention 331 including a chamber 312, a proximal end cap 313 and a distal end cap 314 at the end of a delivery channel 315.

In section view FIG. 32, we see the chamber 312 contains a matrix 321. In an embodiment, matrix 321 consists of a dry porous sponge of matrix material. In an embodiment, the matrix material is substantially collagen, such as soluble Type I collagen. The chamber is sealed at the proximal end by an elastomeric septum/piston 322 and at the distal end by distal end cap 314 and the chamber containing the matrix is evacuated of all air during manufacture.

FIG. 33 shows activation of the matrix with a biologically active agent to form a therapeutic composition. In an embodiment a syringe containing a liquid biologically active agent 331 fitted with a needle 332 is inserted into elastomeric septum/piston 322, thereby creating a passageway in communication with the chamber containing a matrix. A biologically active agent is either injected or simply drawn into the matrix by the vacuum within the chamber. The biologically active agent disperses into the matrix and activating the therapeutic composition.

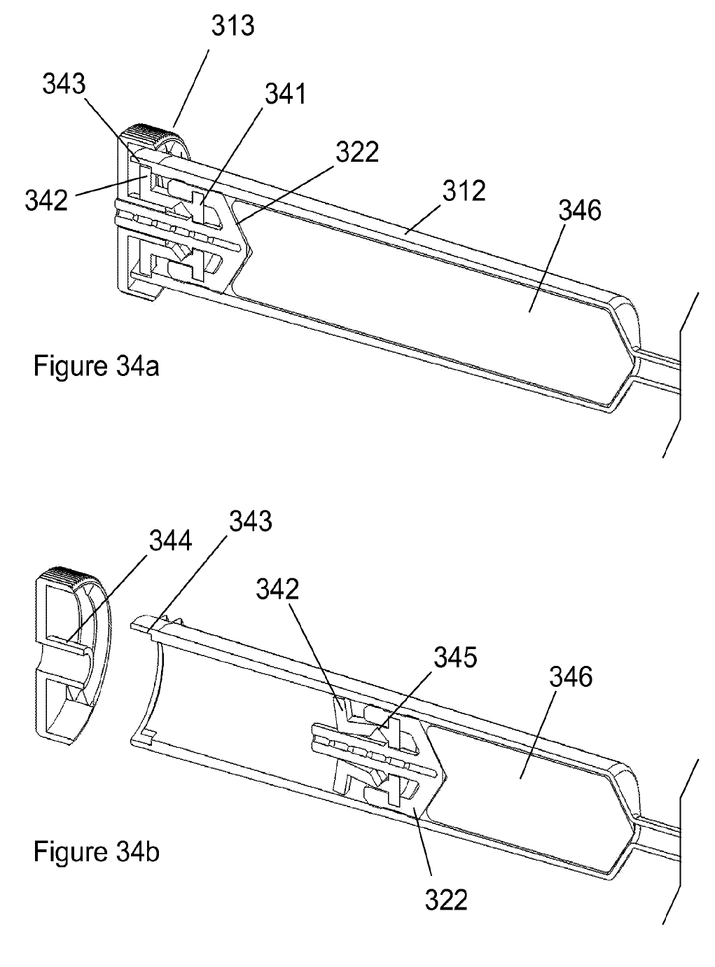

FIG. 34a shows syringe 331 and needle 332 removed and the passageway created through elastomeric septum/piston 322 re-closed, leaving the chamber containing an activated therapeutic composition 346 and empty pores under vacuum. Elastomeric septum/piston 322 is held in position near the proximal end of the device by a latching mechanism 341 with a finger 342 engaging a notch 343 in the wall of chamber 312.

FIG. 34b shows proximal end cap 313 removed. The cap includes a protuberance 344 that holds fingers 342 in notch 343 when attached to the chamber. With the cap unscrewed and removed from the chamber, a hinge 345 allows fingers 342 to swing inward, disengaging notches 343 and allowing translational movement of the elastomeric septum/piston 322. The activated therapeutic composition 346 within the chamber is porous, compressible and under vacuum. The free sliding septum/piston 322 responds to the pressure differential between the vacuum inside the chamber and atmospheric pressure outside by sliding into the therapeutic composition, collapsing the empty pores and compressing the composition until the pressure inside the chamber equalizes with atmospheric pressure outside. The final volume of the therapeutic composition equal to the solid volume of the matrix (less empty pore volume) plus the volume of biologically active agent added. The compressed volume contains virtually no air bubbles.

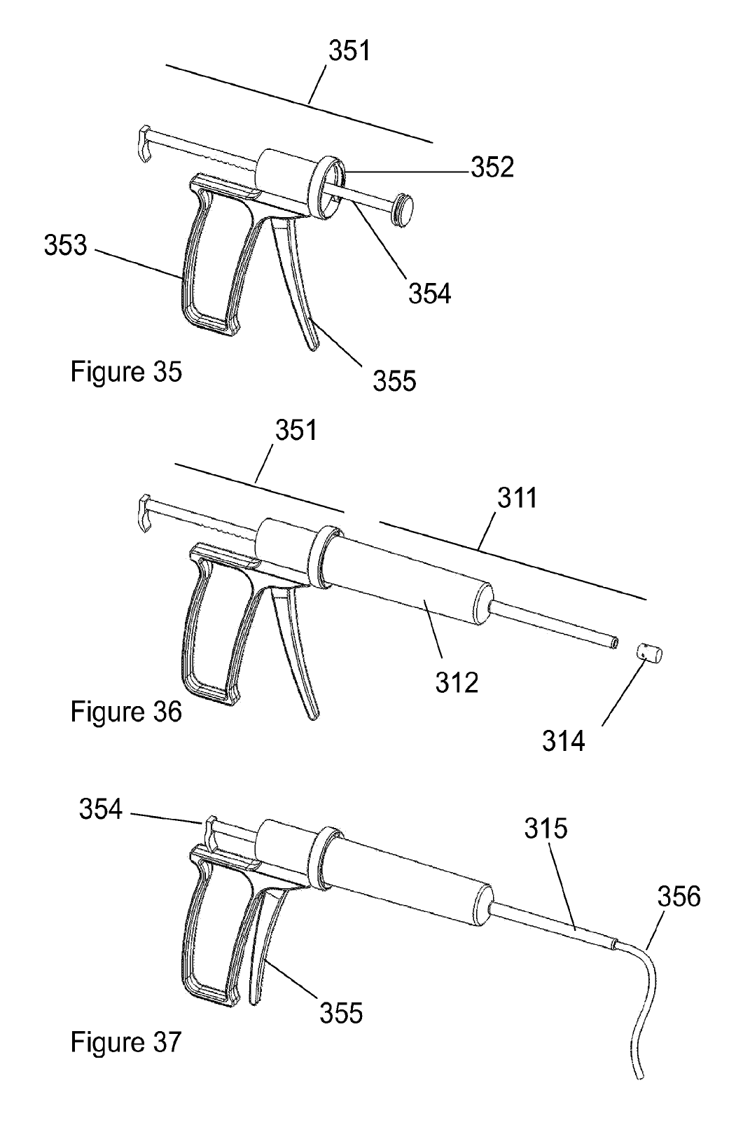

FIG. 35 shows another embodiment of the present invention comprising a body 351 with a screw thread connection element 352 for connecting the body 351 to device 311. In the illustrated embodiment, a screw thread connection element 352, a handle 353, a ratcheted plunger 354, and a trigger 355 act on the plunger 354, such that squeezing the trigger 355 advances the plunger 354 distally (to the right) a small amount. A ratchet pawl and spring (not shown) prevent backward movement of the plunger 354 and return the trigger 355 so the process can be repeated over and over.

FIG. 36 shows the embodiment of the invention assembled and ready for use. Chamber 312 of device 311 screws onto component 351 with plunger 354 pushing against septum/piston 322 (not shown in this view). Distal end cap 314 is removed prior to use.

FIG. 37 shows the device in use. Delivery channel 315 is inserted into a body (not shown) with tissue to be treated. Trigger 355 is repeatedly squeezed and released, causing plunger 354 to advance and push against septum/piston 322 (not shown in this view) which in turn extrudes activated therapeutic composition through the delivery channel and into the body. The delivered therapeutic composition 356 is free of entrapped air and does not sputter when extruded, is incompressible and therefore resistant to backflow of pressurized irrigation fluid (e.g. saline in arthroscopy), and is neutrally buoyant and does not float to the top of the fluid in the body.

1.-23. (canceled)

24. An activation device for a therapeutic compound, comprising:
an outer element defining an interior chamber and including a dry porous collagen matrix disposed therein,
wherein the outer element defines the chamber to be closed and fully enclosing the matrix, and
wherein the chamber is characterized by a static pressure above or below ambient, and wherein the outer element is adapted to receive a delivery element for infusing said matrix with a biologically active agent.

25. A therapeutic kit for a therapeutic compound, comprising:
A. an activation device according to claim 24, and
B. an introducing device adapted to introduce a fluid biologically active agent through the outer element and into the matrix.

26. An activation device for a therapeutic compound comprising:
an outer element defining a cylindrical interior chamber extending along a central axis, the chamber having a piston disposed at one end thereof, and being sealed at the other end thereof, the chamber including a dry porous matrix disposed therein,
further including a selectively operative latch assembly, operative in a first state to fixedly hold the piston in the chamber, and operable in a second state to release the plunger allowing motion of the plunger in the chamber along the central axis in response to a pressure differential across the plunger, and
wherein the chamber is characterized by a static pressure below ambient, and
wherein the piston is adapted to receive a delivery element for infusing the chamber with a biologically active agent.

27. (canceled)

28. A delivery kit for delivering a therapeutic compound including a cylindrical, suture-bearing, dry porous matrix composed of a collagen matrix, comprising:
A. an outer elongated element having a length L1 and extending from a proximal end to a distal end along a delivery axis, and defining an inner cylindrical region,
B. an inner elongated element having a length L2, where L2 is greater than L1, and disposed slidably within the inner cylindrical region of the outer elongated element, and extending from a proximal end to a distal end along the delivery axis, and defining an inner cylindrical region having diameter D1,
C. a pusher assembly extending along a pusher axis from a matrix pusher element at a proximal end to a control pusher element at a distal end,
wherein at least the matrix pusher element at the distal end is adapted to be slidable within the inner cylindrical region of the inner elongated element with the pusher axis being substantially coaxial with the delivery axis, wherein the matrix pusher element extends transverse to the pusher axis and wherein the pusher element has a circumferential edge has a diameter slightly less than D1 and includes a plurality of void regions extending radially inward from the circumferential edge, and
wherein the control pusher element is rigidly coupled to the matrix pusher element by an intermediate element, and
D. a plurality of elongated suture capture devices, each suture capture device extending along a suture device axis from a handle portion at a proximal end to a suture capture portion at a distal end, and having a length greater than L2.

29. A therapeutic composition containment device comprising:
A. an elongated primary shaft extending from a proximal end to a distal end along a shaft axis, defining a primary interior region extending along said shaft axis from said proximal end to said distal end,
B. a containment structure extending from said distal end of said elongated shaft and having two states:
i. a first state wherein said containment structure is C-shaped and disposed about a central axis transverse to said shaft axis, and extends between opposite ends thereof and defining a gap between said opposite ends, and
ii. a second state wherein said containment structure is ring-shaped and disposed about said central axis, said ring-shaped containment structure having an innermost surface defining an open faced circular channel extending circumferentially about said central axis, said channel being in fluid communication with said interior region of said shaft.

30. (canceled)

31. A containment device according to claim 29, wherein said device includes a state control assembly selectively operable from said proximal end to control said containment structure to be in said first state with said gap being substantially closed and in said second state with said gap being opened and having a predetermined non-zero value.

32. A containment device according to claim 31, further comprising:
an outer elongated shaft disposed along an outer axis and defining an interior region extending along said outer axis, said outer elongated shaft being adapted to overfit said primary elongated shaft with said outer axis substantially parallel with said shaft axis, whereby said primary elongated shaft slides within said outer elongated shaft, and
wherein said containment structure is compressible to permit positioning within said interior region of said outer elongated shaft.

33. An apparatus for loading a therapeutic composition, said therapeutic composition including a matrix and a biologically active agent (BAA), comprising:
A. a delivery device, defining an interior cylindrical mixing chamber extending along a central axis between a proximal end and a distal end, and including at said distal end, a delivery tube extending therefrom along said central axis from said distal end to a delivery tip, said delivery tube defining an interior region extending along said central axis and having length L and a minimum dimension D transverse to said central axis and being in fluid communication with said Interior mixing chamber,
B. an activation assembly including a housing having a vacuum (VAC) port, a biologically active agent (BAA) port, and a loading port and a valve, wherein said VAC port, said BAA port and said loading port are coupled to said valve, wherein said valve is operative in a first state to couple said VAC port to said loading port, and in a second state, to couple said BAA port to said loading port, and
C. an elongated mixing tube coupled to and extending from said loading port, said mixing tube defining an interior BAA flow region extending along a BAA loading axis between said loading port and a distal end of said mixing tube.

34. An apparatus according to claim 33, wherein the length of said BAA flow region is greater than L, and wherein said mixing tube includes a plurality of radially extending holes disposed near said distal end of said mixing tube, said holes coupling said BAA flow region to points outside said mixing tube, wherein the maximum diameter of said mixing tube is less than D.

35. An apparatus according to claim 33, further comprising a vacuum supply coupled to said VAC port.

36. An apparatus according to claim 33, further comprising a BAA reservoir coupled to said BAA port.

37. An apparatus according to claim 33, further comprising a piston disposed in said cylindrical mixing chamber and an associated driver, said piston being adapted for motion along said central axis in response to user-controlled action on said driver.

38. An apparatus according to claim 33, further comprising a seal at said loading port for receiving said delivery tip, whereby said interior region of said delivery tube is in fluid communication with said loading port, with said mixing tube disposed within said delivery tube with said central axis being substantially coaxial with said BAA loading axis.

39. An apparatus for loading a therapeutic composition, said therapeutic composition including a matrix and a biologically active agent (BAA), to a delivery device whereby said matrix and said BAA are intermixed.

40. (canceled)

40.-42. (canceled)
